(19) United States
(12) Patent Application Publication
(10) Pub. No.: US 2025/0057458 A1
(43) Pub. Date: Feb. 20, 2025

(54) ELECTRICALLY CONDUCTIVE YARN AND FABRIC-BASED, NOISE-CANCELLING, MULTIMODAL ELECTRODES FOR PHYSIOLOGICAL MEASUREMENTS

(71) Applicant: NEXTILES, INC., Brooklyn, NY (US)

(72) Inventor: George SUN, Brooklyn, NY (US)

(21) Appl. No.: 18/565,233

(22) PCT Filed: Jun. 2, 2022
(86) PCT No.: PCT/US2022/031909

§ 371 Date: Nov. 29, 2023

Related U.S. Application Data
(60) Provisional application No. 63/196,604, filed on Jun. 3, 2021.

(51) Int. Cl. A61B 5/27; A61B 5/00; D02G 3/44; D03D 15/533; D05C 17/00

(52) U.S. Cl. CPC A61B 5/27; A61B 5/6804; D02G 3/441; D03D 15/533; D05C 17/00; A61B 2562/125; D05D 2209/00; D05D 2303/40

(57) ABSTRACT

A system of fibers, filaments, and/or other electrically conductive materials forms an electrically conductive-to-semi-conductive yarn that can be assembled into a textile for measurement of voltage, current, resistance, capacitance, inductance, RF, and/or EM signals. Textiles are formed through weaving, knitting, lacing, and/or non-woven mechanical methods of yarn-making into 2D/3D structures. Textile-based electrodes can be formed via folding, cutting, layering, sewing, and/or embroidering patterns to control signal transmission within/through the electrode. Multiple electrodes are positioned on a surface (e.g., a body) to sequentially or simultaneously perform multiple diagnostic modalities (e.g., electrocardiography, electromyography, electrooculography, electroencephalogram, bioelectrical impedance analysis, skin impedance analysis, and/or electrodermal activity). These modalities are multiplexed using an optimized electrode set through amplitude and frequency deconvolution and filtering algorithms to minimize the quantity of electrodes and connections on the surface while maximizing signal-to-noise ratio, differential and common mode noise rejection, and elimination of external signals (e.g., RF and EM noise).

### CROSS-REFERENCE TO RELATED APPLICATION

This application claims priority to U.S. Patent Application Ser. No. 63/196,604, filed on Jun. 3, 2021, the disclosure of which is incorporated by reference herein in its entirety.

### TECHNICAL FIELD

The presently disclosed subject matter is directed to flexible, fabric-based, noise-cancelling, and multimodal electrodes for physiological measurements. More particularly, the presently disclosed subject matter is directed to a universal electrode material that can reliably detect and measure signals from any desired location on the body; creating an in-garment connector strategy that bonds the electrodes to a CPU within a piece of clothing; and a CPU that is capable of differentiating between the various modes.

### BACKGROUND

Skin-voltage pickup, along with impedance, capacitance, inductance, and current sensing, is one of the most holistic and non-invasive methods to monitor physiological health. Signals from tissue or organs, such as the heart, muscle, eye, and brain, manifest on (e.g., are detectable on) the skin surface as electrical signals. The electrical signal associated with each of the tissue or organ, as the case may be, has an electrical pulse that is unique from signals associated with other tissues or organs. Such electrical signal can be analyzed and mapped (e.g., characterized) after acquisition from the skin. Measuring one or more of these electrical signals can give clinicians, doctors, coaches, healthcare workers, and even at-home users (e.g., patients, or subjects) insight regarding the state of the body from which the electrical signal(s) are acquired. Examples of such body states that can be evaluated through the acquisition and analysis of such electrical signal(s) include determinations regarding fatigue, stress, cognitive load, reflexes, and/or the health of an organ of interest.

At present, multiple skin-voltage measuring modalities are known. One such modality is the electrocardiogram (ECG or EKG) for monitoring heart activity; another is electromyography (EMG) for monitoring muscle activity; another is electrooculogram (EOG) for monitoring eye activity; another is electroencephalography (EEG) for monitoring brain activity. Skin-specific measurements via skin impedance analysis or spectroscopy (SIA) and electrodermal activity (EDA), which measure the skin electrophysiology to monitor nervous system activity, as well as any other chemical or biological changes within the body that may manifest in dermal fluctuations in voltage, current, impedance, capacitance, and/or inductance, are known. Through-skin measurement via bioelectric impedance analysis (BIA), which applies a relatively small electrical current and voltage through the skin, which is then measured by a separate measurement device, are known, such as for calculating, for example, internal body fat, muscle, bone, and/or water content. The aforementioned diagnostic tests can be referred to herein as “modes”.

Each of these measurement techniques require one or more surface electrodes for measuring the skin's electrical characteristics without introducing internal or external noise. These electrodes are tethered to (e.g., using cables) a central processing unit (CPU). The CPU receives raw electric signals from the skin and converts such raw electric signals into a voltage or a graph for analyzing the electrical signal of the skin. BIA is known to require an input signal (e.g., an initial source of voltage and current) that is transmitted through the skin, which is then measured by a second electrode; in BIA, the change in waveform and/or amplitude of the input signal can then be used to calculate one or more aspects regarding body composition.

Each mode differs in the number of electrodes and placement of each electrode on the body. Measurement of heart activity via EKG typically requires 2-10 electrodes, inclusive, with placement on opposite sides of the heart; placement can include locations on the torso, or chest, but can be as far away as the wrists or hands of the user. Measurement of muscle activity via EMG typically requires 2 electrodes, which are typically placed around the muscle of interest. Measurement of eye activity via EOG typically requires 2 electrodes, which are typically placed horizontally between the left and right sides of the eye (for detection of horizontal eye movement) or top and bottom sides of the eye (for detection of vertical eye movement). Measurement of brain activity via EEG typically requires between 4-256 electrodes, inclusive, which are positioned at opposite hemispheres of the skull; the use of more electrodes can be used to increase the resolution of the area and portion of the brain being monitored. Through-skin measurement via BIA typically requires 2 electrodes, which are positioned on opposite sides of the body (e.g., one each at the left and right wrists or one on each foot, which can cover the entire path of the body). Skin-specific measurements via SIA and/or EDA typically require 2 electrodes, which can be positioned at any two points defining the bounds of a segment of skin to be measured.

In each of these modes, the size, shape, and material properties of the electrodes in use in performing such modalities are known to impact quality of the measurement. A suitable electrode is known to advantageously be made from a material having a high electrical conductivity, a low sensitivity to stray resistance, capacitance, and inductance noise, and an impedance compatible with the surface from which measurements are to be obtained. The electrode configuration of such modes can be improved by adding an additional electrode as a reference electrode at a stable location on the body, such as at a bone or large surface area to subtract any noise or variability in the configuration that may affect the primary (e.g., 2) sensing electrodes. The electrodes can also be insulated with non-conductive and/or EMI shielding materials. Control of such variables ultimately impacts the handling and comfort of the electrodes on the patient's body and may cause undesirable side effects, such as moving, twitching, itching, or the patient being in an uncomfortable and agitated state during performance of the mode.

After the electrodes are arranged, connectors are then positioned to transmit signals from the electrodes to the CPU; such connectors are typically snaps or electromechanical clips. Such attachment types are often reversible, such that the electrodes can be taken off and replaced between studies, or such that the connectors can be swapped between electrodes during different studies. Each mode's connector is typically composed of the same material as the electrode, such as a highly conductive cable shielded in a plastic or insulating material. The connectors are electrically connected to a CPU, which is configured to perform the appropriate filtering and analytical operations to produce a result, a graph, or a report. Each mode requires a similar analysis pipeline, such as baselining, noise reduction, and signal amplification. However, each mode may require specific tuning of each analysis stage, as some modes may have a lower intensity electric signal and/or more noise than other modes. Given the complexity of the analysis requirements for each mode, as well as the variability in terms of placement, quantity, and environment in which electrodes are used that may alter the analysis process, each mode typically uses its own specific CPU. In the medical setting, each mode comes with a specific computer and monitor system, which makes portability and accessibility of the measurement difficult and, in some instances, impossible.

The medical field has developed several types of electrodes, wet (or gel) and dry electrodes. Wet or gel electrodes are highly conductive electrodes that are immersed in a liquid interface when contacting the skin. This intermediate liquid is used to provide enhanced conductivity for transfer of the electric signal between the skin and the electrode. The intermediate liquid also aids in impedance matching to ensure the transfer of electrical signal is optimal between the skin and the electrode. Dry electrodes are placed in direct contact with the skin and any intermediate medium (e.g., air, hair, sweat, etc.) is known to reduce the fidelity of the electric signal measured by such electrode. In the medical field, wet electrodes are typically used and replaced between studies or patients. In consumer-directed applications, dry electrodes are often used and users are instructed to wash such electrodes between uses. However, the reliability of measurements obtained from consumer-directed applications is extremely poor and, as such, are unsuitable for health or medical usage (e.g., for diagnostic purposes).

Although the use of such skin-based measurements has been scientifically proven and is credible science, the logistics that are required to configure an experiment and the amount of discomfort a patient must undergo has, ironically, rendered such skin-based measurements one of the hardest diagnostic techniques to perform. Therefore, a need exists at present for creating a universal electrode material that can reliably detect and measure signals from any desired location on the body; creating an in-garment connector strategy that bonds the electrodes to a CPU within a piece of clothing; and developing a CPU that is capable of differentiating between the various modes. The presently disclosed subject matter is also directed towards considerations regarding sizing differences, wearability, and comfort when designing such in-garment electrodes, connectors, and CPUs.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

The following presents a summary to provide a basic understanding of one or more embodiments of the disclosure. This summary is not intended to identify key or critical elements, or to delineate any scope of particular embodiments or any scope of the claims. Its sole purpose is to present concepts in a simplified form as a prelude to the more detailed description that is presented later. In one or more embodiments described herein are systems, methods, and devices that describe various aspects of one or more textile-based electrodes for skin voltage, current, impedance, capacitance, inductance measurement, and other bioelectro-physiological measurement.

According to an example embodiment, a yarn is provided, the yarn comprising: a plurality of polymers, a plurality of fibers, and/or a plurality of filaments; wherein at least some of the plurality of polymers, the plurality of fibers, and/or the plurality of filaments comprise electrically conductive material, electrically semi-conductive material, and/or electrically non-conductive material, whereby the yarn is configured to detect and/or transmit one or more signals in an axial direction or a transverse direction of the yarn, and whereby the yarn is configured to limit susceptibility of the yarn to transmission of a parasitic noise, which parasitic noise is in a form of resistive, capacitive, and/or inductive noise and/or an environmental noise.

In some or all embodiments of the yarn, the plurality of fibers and/or the plurality of filaments are carded and drawn during formation of the yarn such that substantially all of the electrically conductive material and/or substantially all of the electrically semi-conductive material is substantially aligned in a same direction within the yarn to control a response time, an input dynamic range, an output dynamic range, a bandwidth, a signal-to-noise ratio, a common-noise rejection ratio, differential-noise rejection, a signal gain, a sensitivity, and/or an insensitivity of the yarn.

In some or all embodiments of the yarn, the plurality of fibers and/or the plurality of filaments are formed by spinning into continuous strands with a prescribed length, weight, density, tensile strength, and/or pliability.

In some or all embodiments of the yarn, the plurality of fibers and/or the plurality of filaments have a core that comprises the electrically conductive material and/or the electrically semi-conductive material.

In some or all embodiments of the yarn, the plurality of fibers and/or the plurality of filaments comprise the electrically conductive material and/or the electrically semi-conductive material deposited, via chemical vapor deposition (CVD), physical vapor deposition (PVD), electroless deposition, dying, spraying, annealing, and/or coating, on an outer surface of the plurality of fibers and/or an outer surface of the plurality of filaments.

In some or all embodiments of the yarn, the deposition of the electrically conductive material and/or of the electrically semi-conductive material to the plurality of fibers and/or the plurality of filaments is controlled to minimize resistive, capacitive, inductive, radio frequency (RF), and/or electromagnetic (EM) interference, which can be induced by the plurality of fibers and/or the plurality of filaments themselves and/or from an environmental noise source.

In some or all embodiments of the yarn, the yarn is configured to extract and collect parasitic and/or environmental noise from the signals during transmission of the signals through the yarn.

In some or all embodiments of the yarn, the yarn blocks radio frequency (RF) and electromagnetic (EM) interference from the signals during transmission of the signals through the yarn.

In some or all embodiments of the yarn, the yarn is twisted, bulked, dyed, and/or spooled to provide enhanced mechanical properties and such that the yarn reduces motion artifacts in comparison to an untwisted yarn.

According to another example embodiment, a system comprising a plurality of yarns is provided, wherein the plurality of yarns comprises any yarn disclosed herein.

In some or all embodiments of the system, the yarn and one or more other yarns of the plurality of yarns are twisted together and directionally aligned to reduce or reject differential noise and/or common noise.

In some or all embodiments of the system, the plurality of yarns comprise electrically conductive fibers and/or filaments that are twisted with the plurality of yarns and/or electrically conductive polymers deposited on the plurality of yarns to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments of the system, the plurality of yarns are twisted in a Z direction and/or an S direction to form one or more plys, cords, and/or ropes to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments, the system comprises effectors and/or binders that are wrapped around one or more yarns of the plurality of yarns, wherein the effectors and/or binders are electrically conductive, electrically semi-conductive, or electrically non-conductive to control an aesthetic feature, a textural feature, and/or an electrical conductivity of the one or more yarns.

According to another example embodiment, a method of making a yarn is provided, the method comprising: forming, from a plurality of polymers, a plurality of fibers, and/or a plurality of filaments, the yarn; wherein at least some of the plurality of polymers, the plurality of fibers, and/or the plurality of filaments comprise electrically conductive material, electrically semi-conductive material, and/or electrically non-conductive material, such that the yarn can detect and/or transmit one or more signals in an axial direction or a transverse direction of the yarn and also limit susceptibility of the yarn to transmission of a parasitic noise, which parasitic noise is in a form of resistive, capacitive, and/or inductive noise and/or an environmental noise.

In some or all embodiments, the yarn-making method comprises carding the plurality of fibers and/or the plurality of filaments; drawing the plurality of fibers and/or the plurality of filaments; and optionally, aligning, sorting, and/or organizing the plurality of fibers and/or the plurality of filaments; wherein, in carding and drawing the plurality of fibers and/or the plurality of filaments, all of the electrically conductive material and/or all of the electrically semi-conductive material is aligned in a same direction within the yarn to control a response time, an input dynamic range, an output dynamic range, a bandwidth, a signal-to-noise ratio, a common-noise rejection ratio, differential-noise rejection, a signal gain, a sensitivity, and/or an insensitivity of the yarn.

In some or all embodiments, the yarn-making method comprises spinning the plurality of fibers and/or the plurality of filaments into continuous strands with a prescribed length, weight, density, tensile strength, and/or pliability.

In some or all embodiments of the yarn-making method, the plurality of fibers and/or the plurality of filaments have a core that comprises the electrically conductive material and/or the electrically semi-conductive material.

In some or all embodiments, the yarn-making method comprises depositing the electrically conductive material and/or the electrically semi-conductive material deposited on an outer surface of the plurality of fibers and/or on an outer surface of the plurality of filaments via chemical vapor deposition (CVD), physical vapor deposition (PVD), electroless deposition, dying, spraying, annealing, and/or coating.

In some or all embodiments, the yarn-making method comprises controlling the depositing of the electrically conductive material and/or of the electrically semi-conductive material on the outer surface of the plurality of fibers and/or on the outer surface of the plurality of filaments to minimize resistive, capacitive, inductive, radio frequency (RF), and/or electromagnetic (EM) interference, which can be induced by the plurality of fibers and/or the plurality of filaments themselves and/or from an environmental noise source.

In some or all embodiments of the yarn-making method, the yarn can extract and collect noise from the signals during transmission of the signals through the yarn.

In some or all embodiments of the yarn-making method, the yarn can be used as a shield for blocking radio frequency (RF) and electromagnetic (EM) interference from the signals during transmission of the signals through the yarn.

In some or all embodiments, the yarn-making method comprises twisting, bulking, dying, and/or spooling the yarn to enhance one or more mechanical properties and to reduce motion artifacts induced by motion of the yarn in comparison to an untwisted yarn.

According to another example embodiment, a method of making a ply, cord, and/or rope is provided, the method comprising providing a plurality of yarns, wherein the plurality of yarns comprises any yarn disclosed herein. In some or all embodiments, the ply-, cord-, and/or rope-making method comprises twisting together and directionally aligning the yarn and one or more other yarns of the plurality of yarns to reduce or reject differential noise and/or common noise.

In some or all embodiments, the ply-, cord-, and/or rope-making method comprises twisting electrically conductive fibers and/or filaments with the plurality of yarns; and/or depositing electrically conductive polymers on the plurality of yarns; and controlling, as a result of the twisting and/or depositing, a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments of the ply-, cord-, and/or rope-making method, the plurality of yarns are twisted in a Z direction and/or an S direction to form the ply, the cord, and/or the rope to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments, the ply-, cord-, and/or rope-making method comprises wrapping effectors and/or binders one or more yarns of the plurality of yarns, wherein the effectors and/or binders are electrically conductive, electrically semi-conductive, or electrically non-conductive to control an aesthetic feature, a textural feature, and/or an electrical conductivity of the one or more yarns.

According to another example embodiment, an electrically conductive textile-based electrode is provided, the electrode comprising: a textile comprising a plurality of yarns interlaced in horizontal, vertical, and/or angled directions; wherein the plurality of yarns comprises yarns that are electrically conductive, electrically semi-conductive, and/or electrically non-conductive; and wherein the electrode is configured to form and/or control a primary signal path for transmission of signals in an axial direction and/or in a transverse direction.

In some or all of the embodiments of the electrode, the plurality of yarns are formed in repeated or irregular patterns of underlays and overlays that are configured to transmit the signals in a direction of extension of the electrode, as well as on a top surface, internal to, and/or on a bottom surface of the electrode.

In some or all of the embodiments of the electrode, the plurality of yarns are assembled together using a weaving technique, a knitting technique, a lacing technique, and/or a non-woven technique to form the electrode.

In some or all of the embodiments of the electrode, a shape, size, thickness, and/or material type of the electrode can be selected to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

In some or all of the embodiments of the electrode, in forming the electrode, the textile is cut, folded, sewn, embroidered, and/or stacked horizontally and/or vertically to have a series of textile layers that can each be electrically conductive, electrically semi-conductive, and/or electrically non-conductive.

In some or all of the embodiments of the electrode, cutting and/or folding of the textile and/or stacking a series of textile layers horizontally and/or vertically is used to control a primary transmission path for the signals in a direction of extension of the textile and/or in a direction perpendicular to the direction of extension.

In some or all of the embodiments of the electrode, for the textile, ends per inch, picks per inch, stitches per inch, knits per inch, and/or weaves per inch can be selected to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

In some or all of the embodiments of the electrode, for the textile, a weight, a density, a stitch pattern, a ratio of underlay and overlay yarns of the textile and a direction of the signals within the electrode are selected to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

In some or all of the embodiments of the electrode, a stitch pattern of the textile from which the electrode is formed can be selected to control a signal transmission path in which the signals can gain or attenuate measurements comprising voltage, current, resistance, capacitance, and/or inductance.

In some or all of the embodiments of the electrode, a stitch pattern of the textile from which the electrode is formed can be selected to control a signal transmission path in which the signals can disrupt, shield, and/or absorb external noise from radio frequencies, electromagnetic radiation, and/or voltage, current, resistive, capacitive, and/or inductive signals from an adjacent noise source.

In some or all embodiments, the electrode comprises electrically conductive and/or electrically semi-conductive yarns that are embroidered in the textile to control a direction of transmission of the signals within the electrode to aggregate, absorb, or differentially transmit signal and noise sources.

In some or all embodiments, the electrode comprises an electrically conductive and/or electrically semi-conductive yarn that is, by varying a tension applied thereto when being sewn into the textile, at the top surface and/or the bottom surface of the textile to control a direction of transmission of the signals within the electrode and/or an interface with electrically conductive, electrically semi-conductive, and electrically non-conductive regions formed in the textile.

In some or all of the embodiments of the electrode, by cutting or folding the textile and/or by stacking a series of textile layers horizontally and/or vertically, the electrode is configured to maintain at least one area of contact, and/or with a fractal pattern, with a measurement location to ensure sufficient impedance matching for signal transmission.

In some or all of the embodiments of the electrode, the electrode is configured such that the signals can enter or exit the electrode through a textile patch, which is sewn, embroidered, hemmed, crimped, soldered, magnetic, chemical bond, or combinations thereof to the electrode, to connect the electrode with further devices.

In some or all embodiments, the electrode comprises a plurality of horizontally or vertically stacked textile layers formed from the textile, wherein the textile layers are angled such that the horizontal and vertical yarns create looped patterns or pores between the textile layers to form the electrode.

In some or all embodiments, the electrode comprises a plurality of horizontally or vertically stacked textile layers formed from the textile, wherein the textile layers are configured to control a resistive signal, a capacitive signal, and/or an inductive signal through a transverse direction of the electrode.

In some or all embodiments, the electrode comprises a plurality of horizontally or vertically stacked textile layers formed from the textile, wherein the textile layers are knitted, woven, sewn, and/or electromechanically and/or chemically attached to secure edges of the electrode in repeating patterns, thereby controlling signal transmission within the electrode.

In some or all of the embodiments of the electrode, the textile is embroidered, folded, cut, and/or stacked with an additional textile layer configured as a signal reservoir and/or a sacrificial textile layer for absorbing noise.

In some or all of the embodiments of the electrode, the textile is embroidered, folded, cut, and/or stacked with an additional textile layer for impedance matching with a measurement location to optimize power transmission into and/or out of the electrode.

According to another example embodiment, a method of forming an electrically conductive textile-based electrode is provided, the method comprising: interlacing a plurality of yarns in horizontal, vertical, and/or angled directions to form a textile, wherein the plurality of yarns comprises yarns that are electrically conductive, electrically semi-conductive, and/or electrically non-conductive; forming and/or controlling a primary signal path within the electrode; and transmitting signals along the primary signal path in an axial direction and/or in a transverse direction; wherein the primary signal path is controlled such that the primary signal path passes through or adjacent to electrically non-conductive regions of the textile and electrically conductive regions of the textile to reduce noise and/or reject transmission of differential and/or common noise; and optionally, wherein the electrically non-conductive regions and the electrically conductive regions of the textile absorb, in the manner of a reservoir, noise introduced along the primary signal path.

In some or all embodiments, the electrode-forming method comprises forming the plurality of yarns in repeated or irregular patterns of underlays and overlays; and transmitting, via the underlays and overlays, the signals in a direction of extension of the electrode, as well as on a top surface and/or on a bottom surface of the electrode.

In some or all embodiments, the electrode-forming method comprises assembling the plurality of yarns together using a weaving technique, a knitting technique, a lacing technique, and/or a non-wove technique to form the electrode.

In some or all embodiments, the electrode-forming method comprises selecting a shape, size, thickness, and/or material type of the electrode to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

In some or all embodiments, the electrode-forming method comprises, while forming the electrode, cutting, folding, sewing, embroidering, and/or stacking the textile horizontally and/or vertically to have a series of textile layers that can each be electrically conductive, electrically semi-conductive, and/or electrically non-conductive.

In some or all embodiments of the electrode-forming method, cutting and/or folding of the textile and/or stacking a series of textile layers horizontally and/or vertically is used to control a primary transmission path for the signals in a direction of extension of the textile and/or in a direction perpendicular to the direction of extension.

In some or all embodiments, the electrode-forming method comprises selecting, for the textile, ends per inch, picks per inch, stitches per inch, knits per inch, and/or weaves per inch to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

In some or all embodiments, the electrode-forming method comprises selecting, for the textile, a weight, a density, a stitch pattern, a ratio of underlay and overlay yarns of the textile and a direction of the signals within the electrode to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

In some or all embodiments, the electrode-forming method comprises selecting a stitch pattern of the textile from which the electrode is formed to control a signal transmission path in which the signals can gain or attenuate measurements comprising voltage, current, resistance, capacitance, and/or inductance.

In some or all embodiments, the electrode-forming method comprises selecting a stitch pattern of the textile from which the electrode is formed to control a signal transmission path in which the signals can disrupt, shield, and/or absorb external noise from radio frequencies, electromagnetic radiation, and/or voltage, current, resistive, capacitive, and/or inductive signals from an adjacent noise source.

In some or all embodiments, the electrode-forming method comprises embroidering electrically conductive and/or electrically semi-conductive yarns in the textile to control a direction of transmission of the signals within the electrode to aggregate or differentially transmit signal and noise sources.

In some or all embodiments, the electrode-forming method comprises varying a tension applied to an electrically conductive and/or electrically semi-conductive yarn that is sewn into the textile, at the top surface and/or the bottom surface of the textile to control a direction of transmission of the signals within the electrode and/or an interface with electrically conductive, electrically semi-conductive, and electrically non-conductive regions formed in the textile.

In some or all embodiments, the electrode-forming method comprises maintaining, by cutting or folding the textile and/or by stacking a series of textile layers horizontally and/or vertically, at least one area of contact, optionally, with a fractal pattern, with a measurement location to ensure sufficient impedance matching for signal transmission.

In some or all embodiments, the electrode-forming method comprises transmitting the signals into or out of the electrode through a textile patch, which is sewn, embroidered, hemmed, crimped, soldered, magnetic, chemical bond, or combinations thereof to the electrode, to connect the electrode with further devices.

In some or all embodiments, the electrode-forming method comprises forming textile layers from the textile; stacking, horizontally or vertically, the textile layers; and angling adjacent textile layers relative to each other such that the horizontal and vertical yarns create looped patterns or pores between the textile layers to form the electrode.

In some or all embodiments, the electrode-forming method comprises forming textile layers from the textile; stacking, horizontally or vertically, the textile layers; and using the textile layers to control a resistive signal, a capacitive signal, and/or an inductive signal through a transverse direction of the electrode.

In some or all embodiments, the electrode-forming method comprises forming textile layers from the textile; stacking, horizontally or vertically, the textile layers; and knitting, weaving, sewing, and/or electromechanically and/or chemically attaching the textile layers to secure edges of the electrode in repeating patterns, thereby controlling signal transmission within the electrode.

In some or all embodiments, the electrode-forming method comprises embroidering, folding, and/or stacking the textile with an additional textile layer, which is operable as a signal reservoir, and/or a sacrificial textile layer, which is operable for absorbing noise.

In some or all embodiments, the electrode-forming method comprises embroidering, folding, and/or stacking the textile with an additional textile layer for impedance matching with a measurement location to optimize power transmission into and/or out of the electrode.

According to another example embodiment, a system for performance of a plurality of diagnostic modalities is provided, the system comprising: a plurality of the electrodes of any of claims 29-47, the plurality of electrodes being positioned over a surface for simultaneous topographical measurement of signals associated with the plurality of diagnostic modalities; and a processor configured to receive and analyze the signals from the plurality of electrodes; wherein the plurality of diagnostic modalities comprises two or more of electrocardiogram (EKG), electromyography (EMG), electrooculogram (EOG), electroencephalography (EEG), bioelectric impedance analysis (BIA), electrodermal activity (EDA), and skin impedance analysis or spectroscopy (SIA).

In some or all embodiments of the system for performance of a plurality of diagnostic modalities, electrodes in the plurality of electrodes comprise different sizes to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments of the system for performance of a plurality of diagnostic modalities, electrodes in the plurality of electrodes comprise different shapes to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments of the system for performance of a plurality of diagnostic modalities, respective placements of electrodes in the plurality of electrodes on the surface can be selected to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments of the system for performance of a plurality of diagnostic modalities, one or more electrodes of the plurality of electrodes are cut, folded, nicked, sewn, embroidered, and/or adhered such that the one or more electrodes maintain sufficient contact with the surface on which the one or more electrodes are positioned while in motion to maintain signal transfer and impedance matching between the one or more electrodes and the surface.

In some or all embodiments of the system for performance of a plurality of diagnostic modalities, one or more electrodes of the plurality of electrodes are cut, folded, nicked, sewn, embroidered, and/or adhered such that that the one or more electrodes expose redundant surface patterns to allow multiple signals to be received at that the one or more electrodes simultaneously.

In some or all embodiments of the system for performance of a plurality of diagnostic modalities, an electrode shape, an electrode thickness, an electrode position, an electrode placement, and/or a distance between adjacent electrodes of the plurality of electrodes is selectable to control a sensitivity of an electrode of the plurality of electrodes to receive and transmit multiple signals.

In some or all embodiments of the system for performance of a plurality of diagnostic modalities, an electrode shape, an electrode thickness, an electrode placement over the surface, and/or a distance between adjacent electrodes of the plurality of electrodes is selectable to control a sensitivity of an electrode of the plurality of electrodes to receive at least at a Nyquist frequency of the signals.

In some or all embodiments of the system for performance of a plurality of diagnostic modalities, the plurality of electrodes define an electrode set, which is optimizable for reducing a quantity of the plurality of electrodes, a placement of the plurality of electrodes over the surface, a size of the plurality of electrodes, a shape of the plurality of electrodes, a thickness of the plurality of electrodes, and/or a connection type of the plurality of electrodes to the surface to measure multiple signals simultaneously.

In some or all embodiments of the system for performance of a plurality of diagnostic modalities, a quantity of electrodes in the plurality of electrodes can be minimized by determining a vector of a start and end electrode that can measure a shared direction for each measurement of the signals.

In some or all embodiments of the system for performance of a plurality of diagnostic modalities, the plurality of electrodes define an electrode set, which is optimizable using feature optimization and predictive and generative algorithms with machine learning models in categories of supervised, unsupervised, semi-supervised, and/or reinforced models.

In some or all embodiments of the system for performance of a plurality of diagnostic modalities, the plurality of electrodes define an electrode set, which is connected to the processor, which is configured to filter and deconvolve signals to extract signals for performance of the plurality of diagnostic modalities from a same signal.

In some or all embodiments of the system for performance of a plurality of diagnostic modalities, the plurality of electrodes define an electrode set, which is connected to the processor, which is configured to transfer signals in a wired and/or wireless manner to a receiving device for storage, processing, and/or machine learning.

In some or all embodiments of the system for performance of a plurality of diagnostic modalities, the plurality of diagnostic modalities are performed simultaneously using a same signal.

According to another example embodiment, a garment is provided, the garment comprising any system for performance of a plurality of diagnostic modalities as disclosed herein, optionally wherein the garment is wearable by a subject, e.g. a human or animal subject.

In some or all embodiments of the garment, the plurality of electrodes are formed in a unitary, undivided, and continuous manner with the garment.

According to another example embodiment, a method of manufacturing a system for performance of a plurality of diagnostic modalities, the system comprising: a plurality of the electrodes as disclosed herein, the plurality of electrodes being positioned over a surface for simultaneous topographical measurement of signals associated with the plurality of diagnostic modalities;

and a processor configured to receive and analyze the signals from the plurality of electrodes; wherein the plurality of diagnostic modalities comprises two or more of electrocardiogram (EKG), electromyography (EMG), electrooculogram (EOG), electroencephalography (EEG), bioelectric impedance analysis (BIA), electrodermal activity (EDA), and skin impedance analysis or spectroscopy (SIA).

In some or all embodiments, the system-manufacturing method comprises selecting a different size for some electrodes in the plurality of electrodes than for other electrodes in the plurality of electrodes to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments, the system-manufacturing method comprises selecting a different shape for some electrodes in the plurality of electrodes than for other electrodes in the plurality of electrodes to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments, the system-manufacturing method comprises selecting placements of electrodes in the plurality of electrodes on the surface to control a response time, an input dynamic range, an output dynamic range, a bandwidth, a signal-to-noise ratio, a common-noise rejection ratio, differential-noise rejection, a signal gain, a sensitivity, and/or an insensitivity of the system.

In some or all embodiments, the system-manufacturing method comprises cutting, folding, nicking, sewing, embroidering, and/or adhering one or more electrodes of the plurality of electrodes such that the one or more electrodes maintain sufficient contact with the surface on which the one or more electrodes are positioned while in motion to maintain signal transfer and impedance matching between the one or more electrodes and the surface.

In some or all embodiments, the system-manufacturing method comprises cutting, folding, nicking, sewing, embroidering, and/or adhering one or more electrodes of the plurality of such that that the one or more electrodes expose redundant surface patterns to allow multiple signals to be received at that the one or more electrodes simultaneously.

In some or all embodiments, the system-manufacturing method comprises selecting one or more of an electrode shape, an electrode thickness, an electrode position, an electrode placement, and/or a distance between adjacent electrodes of the plurality of electrodes to control a sensitivity of an electrode of the plurality of electrodes to receive and transmit multiple signals.

In some or all embodiments, the system-manufacturing method comprises selecting one or more of an electrode shape, an electrode thickness, an electrode placement over the surface, and/or a distance between adjacent electrodes of the plurality of electrodes to control a sensitivity of an electrode of the plurality of electrodes to receive at least at a Nyquist frequency of the signals.

In some or all embodiments of the system-manufacturing method, the plurality of electrodes define an electrode set, the method comprising optimizing the electrode set to measure multiple signals simultaneously, wherein optimizing the electrode set comprises one or more of reducing a quantity of the plurality of electrodes, selecting a placement of the plurality of electrodes over the surface, selecting a size of the plurality of electrodes, selecting a shape of the plurality of electrodes, selecting a thickness of the plurality of electrodes, and/or selecting a connection type of the plurality of electrodes to the surface.

In some or all embodiments, the system-manufacturing method comprises determining a vector of a start and end electrode that can measure a shared direction for each measurement of the signals to minimize a quantity of electrodes in the plurality of electrodes.

In some or all embodiments of the system-manufacturing method, the plurality of electrodes define an electrode set, the method comprising using feature optimization and predictive and generative algorithms with machine learning models in categories of supervised, unsupervised, semi-supervised, and/or reinforced models to optimize the electrode set.

In some or all embodiments of the system-manufacturing method, the plurality of electrodes define an electrode set, which is connected to the processor, the method comprising filtering and deconvolving signals to extract signals for performance of the plurality of diagnostic modalities from a same signal.

In some or all embodiments of the system-manufacturing method, the plurality of electrodes define an electrode set, which is connected to the processor, the method comprising transferring signals in a wired and/or wireless manner to a receiving device for storage, processing, and/or machine learning.

In some or all embodiments, the system-manufacturing method comprises performing the plurality of diagnostic modalities simultaneously using a same signal.

These and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

FIG. 1 is a schematic illustration of steps for a method of making yarn.

FIGS. 2A and 2B show the stages in which fibers are made, and at what process steps conductive material(s) can be introduced into the fibers to form an electrically semi-conductive or electrically conductive yarn.

FIGS. 3A-3M shows the different types of yarns that can be formed using fibers or filaments, and how yarns can be further modified through mechanical post-processing.

FIGS. 4A-4E show examples of different options and combinations of materials that can comprise a conductive yarn.

FIGS. 5A-5G show further example embodiments of yarns that can be produced in the spinning step, such that electrically conductive, semi-conductive, or non-conductive yarn can be made using different combinations of electrically conductive, semi-conductive, and/or non-conductive fibers and/or filaments.

FIGS. 6A-6C show example embodiments regarding the effect of the direction, rate of twisting, and ratio of twisted fibers or filaments around each other or a core can imbue the resultant yarn formed with advantageous mechanical and/or electromechanical properties.

FIGS. 7A-7D show examples of how twisting fibers, filaments, and/or yarns can be used to create secondary strand structures.

FIG. 8A shows example embodiments of a yarn with a core, which can be electrically conductive, semi-conductive, or non-conductive, that is twisted with a material that is electrically conductive, semi-conductive, or non-conductive.

FIG. 8B shows two example embodiments in which, respectively, the use of straight or twisted fibers or filaments can be selected to affect and/or control the resistance, capacitance, and/or inductance of the yarn at the level of the fiber and/or filament.

FIGS. 9A-9D show how signals travel through the axial or transverse direction of a textile made of horizontal and vertical interlacing of conductive, semi-conductive, and non-conductive yarns.

FIGS. 10A-10D show cross-sectional views of textiles made through a woven, knitted, laced, and/or non-woven process.

FIGS. 11A-11E show cross-sectional views of example embodiments of textiles that can act as electrodes to receive and transmit signals from the surface of a system.

FIGS. 12A-12C show how different yarn and textile types may be utilized in an electrode to optimize for signal-to-noise ratio for signal transmission.

FIG. 13 is a graphical representation of how a single electrode can differentially measure signals received from the body.

FIGS. 14A-14C show example embodiments for groups of electrodes in a system that may be organized into a set of electrodes to enable multiplex detection of multiple skin-voltage measurements on the human body at the same time

FIGS. 15A and 15B show example embodiments for the components of a measurement system, as well as the pipeline in acquiring, filtering, storing, and processing received data.

FIG. 16 shows an example embodiment of a flowchart for the data transformation and processing steps that allow an electrode to extract and distinguish specific signals from multiple modalities.

FIG. 17 shows an example embodiment of a flowchart for regarding how a single electrode can be used to simultaneously extract a signal containing information pertinent with the performance of multiple modalities from a raw data source.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

A general set of definitions for some terminology used in the instant application is as follows. The terms fiber and filament are given their typical, customary definition. A yarn is a structure made from linear assemblies of fibers and filaments. A thread is a yarn specifically made for sewing and embroidery. Interlaced yarn can be used to create a fabric (e.g., a woven, knitted, laced, or non-woven fabric). A textile is the general term used to reference an interlaced assembly of yarns. A fabric is used to refer to textiles used for a finished good and can be cut and sewn with the intention of incorporating into such a finished good (e.g., a garment). An electrode is used to refer to a conductive textile (or fabric, if specifically designed for a garment) used for measuring and transmitting electrical signals. When discussing an electrode in the context of a textile, an electrode assembly is used to refer to one or more electrodes assembled to create a single electrode having, for example, enhanced physical, aesthetic, and/or signal transmission characteristics. The term optimized electrode set is used to refer to a group of electrodes that are organized to optimize for the minimal distance covered and the minimal quantity of electrodes, with the highest sensitivity and signal-to-noise ratio (SNR) for the diagnostic modes to be performed. A mode, or modality is used to refer to a study of, or field of, measurement. Examples of such modes include EKG, EMG, EOG, etc. In the context of a mode or modality, the term signal is used to refer to the specific value that is generated when executing a specified mode or modality; the term measurement is used to refer to an instance a device (e.g., electrode) records, detects, senses, or otherwise picks up a signal. The term system is used as a general term for the area in which electrodes are placed for measurement, with the term body typically referring to a corporeal form of a lifeform, such as a human or animal body, or a body that produces electrical signals. The term noise can be used, for example, to refer to instances in which a signal is received that is not the result of the biological process or activity being observed by execution of the mode; examples of such noise can be resistive, capacitive, inductive, radio frequency (RF), electromagnetic (EM), mechanical, and motion artifacts. Intrinsic noise is generated from within the sensing device (e.g., electrode). External noise is generated from the environment and/or system. Noise shielding, absorbing, and/or sacrificial layers may be used to decrease or reduce intensity of signal noise.

Textiles are any fiber, filament, or yarn that can be made into a fabric or cloth. Fibers are thin, small units that are spun together to produce a yarn. Filaments are similar to fibers but are categorized as having extreme lengths in relationship to their diameter, typically a length that is at least 100 times greater than a diameter or width. Fibers can be produced, for example, from organic materials, including cotton, silk, wool, flax, bamboo, and other animal or plant-based materials. Fibers and filaments can also be made of synthetic materials, such as polyester, nylon, rayon, acrylic, and/or metal(s). Due to the nature of synthetic fiber manufacturing, synthetic fibers can be extruded, packed, or pulled with extreme lengths to produce filaments that can act as a core for a yarn; synthetic fibers can also be spun and woven like organically derived fibers.

Yarns are composed of straightened, spun, and/or twisted mixtures of fibers or filaments. Organic or synthetic fibers can be assembled and spun to make a yarn. A combination of organic and synthetic fibers may be referred to as a semi-synthetic yarn. Steps that proceed yarn creation include, for example, raw material sourcing, fiber creation, cleaning, consolidation, and/or shipping of fibers to designated facilities. Multiple processing steps are needed to prepare fibers into a polymeric spun chain that forms a yarn. Fibers are first carded and drawn to orient each fiber in the same direction and to smooth any unnecessary twists or bundles. Once drawn, fibers are spun to form polymeric chains of connections with other fibers to be combined in a single monofilament yarn. The yarn is then wound into a spool where it is easily collected, handled, and/or transferred to the next processing step, which may include spinning into smaller spools, dying, and/or packaging. Yarns can also be wound into small bobbins for sewing and/or storage purposes.

Once a yarn is made, secondary processing steps may be performed, such as twisting (also known as plying), untwisting, bulking, and/or further refined spinning to form a more uniform aesthetic or texture with additional mechanical properties. When yarns are twisted, a ply is formed. Singly twisted yarns can go in the right-handed or left-handed direction, also known as the Z or S direction, respectively. Doubly twisted yarns may be referred to as two-ply, triply twisted yarns may be referred to as three-ply, etc. The benefits of twisting yarns to form a ply are stronger, more mechanically resistant yarns, as well as improved aesthetic features such as texture, feel, friability, and thickness. Yarns can also be stylized with binders and/or effectors to further add physical traits such as added feel or aesthetic changes.

Threads are special types of yarns that are manufactured to be used for sewing or embroidery, which are typically stronger, more taut, and capable of mechanically holding two or more pieces of fabric together. Threads are typically produced as twisted yarns, such as two-ply or three-ply.

Multiple twisted yarns are used to form a cord. Large pieces of cord form a rope. Cords and ropes are typically used for non-sewing purposes but can sometimes be incorporated into worn garments. Examples include structural or mechanical applications, such as tying materials together, forming large canvases of fabrics, bedding, infrastructure, and/or non-worn garments, as these dense yarns may be too heavy and uncomfortable to be worn by a human or animal subject.

According to the presently disclosed subject matter, at each stage of the yarn-making process, one or more subprocesses are performed, including, for example, the incorporation of metallic materials to produce electrically conductive to semi-conductive yarns. Fibers or filaments can be combined with metallized materials, such as polymeric or organic materials through extrusion, pulling, deposition, annealing, electrolysis, and/or other electrochemical and mechanical processes. Such electrically conductive materials are then incorporated into the yarn-making process to produce a semi-conductive to conductive yarn (e.g., a yarn that has an electrical conductivity between semi-conductive and conductive).

Examples as to how electrically conductive materials can be incorporated into the fiber, filament, and yarn-making process to create conductive yarns are included hereinbelow, as follows. In one example embodiment, one or more fibers of electrically non-conductive and one or more fibers of electrically conductive materials are carded and drawn together before being spun into a yarn, thereby forming a yarn having a composition of a mixture of electrically non-conductive/conductive fibers. In another example embodiment, one or more filaments of electrically non-conductive material and one or more filaments of electrically conductive material are spun into a yarn. In another example embodiment, a pre-spun electrically non-conductive yarn is coated with metallic fibers and/or particles. In another example embodiment, one or more filaments of electrically non-conductive material are first coated with electrically conductive fibers and/or particles and the filaments are then spun into a yarn. In another example embodiment, one or more filaments of electrically conductive material are first coated with non-conductive fibers or particles and then spun into a yarn.

After the semi-conductive to conductive yarn is made, further metallic incorporation can be performed by one or more of the following examples: (1) Twisting an electrically non-conductive yarn around an electrically conductive filament core; (2) Twisting an electrically conductive yarn around an electrically non-conductive filament core; (3) Twisting one or more electrically non-conductive yarns together with one or more electrically conductive yarns; (4) Twisting one or more electrically non-conductive yarns together and depositing electrically conductive fibers and/or particles around the resultant ply.

The degree (e.g., concentration) to which metallic materials (e.g., particles) are incorporated into the yarn determines the yarn's electromechanical properties and the fitness of the yarn for use as a sensor for voltage, current, resistance, capacitance, and/or inductance signal pickup. The electrical properties of the yarn, such as resistance, capacitance, and/or inductance, are tuned (e.g., controlled, or selected) throughout the yarn-making process, as the length, twists, surface area, and distance between fibers impact these electrical signals. Additionally, a change in resistance, capacitance, and/or inductance are related to signal transmission properties, such as signal-to-noise, common mode rejection, sensitivity, response rate, dynamic range, gain, and/or bandwidth.

Examples of the physical characteristics of a yarn that determine the electrical properties of resistance (R), capacitance (C), and inductance (L) of such a yarn are included hereinbelow. The dimensions referenced are based on an assumption that a signal is coming from the axial plane (e.g., through the thread).

The equation that determines resistance of a yarn is

$$R = ρ \frac{l}{A} ,$$

where ρ is resistivity, l is the length of the yarn, and A is the cross-sectional area, or diameter, of yarn.

The equation that determined capacitance of a yarn is

$$C = ϵ \frac{A}{d} ,$$

where ε is an electric constant, permeability (interchangeable with permittivity) to field flux, A is the area of contact of two adjacent yarns, and d is the distance between two adjacent yarns. The electric contact, ε, can be affected by adding a dielectric in between the two conductive parts. There can also exist an intermediate layer between two adjacent yarns that provides an additional dielectric constant, which is multiplied with the electric constant E. The intermediate layer can also be a yarn. κ is the dielectric constant of the intermediate material and κ*ε is the combined permittivity.

The equation that determines inductance of a yarn is

$$L = μ \frac{{N}^{2} \cdot A}{l} ,$$

where μ is the permeability of the material from which the yarn is made, N is the number of continuous twists of the yarn per unit length, A is the area of the yarn, and l is the length of continuous twists of the yarn.

The aforementioned equations will be changed slightly if transmission transversely intersecting through the yarn is accounted for, in which case dimensions such as length and area are measured through the thickness of the yarn and the area is the area of the yarn's body interfacing with the signal.

After having been spooled, yarns can then be assembled in a yarn-by-yarn manner into larger textiles through any suitable interlacing technique, or series of interlacing techniques. The most common such interlacing techniques are weaving and knitting. Weaving is used to form a structured two-dimensional (2D) array of yarns woven together in the horizontal and vertical directions. Knitting uses one or more yarns to create stitches, or loops, that are assembled in a cartesian or polar (e.g., circular) direction to form a fabric. There also exist other interlacing techniques, which incorporate heterogeneous mixtures of yarns to form patterns that have yarn patterns that are from loose-to-dense and are free to go in angled directions in the textile. Other techniques that may be used are referred to herein as non-woven processes. Non-woven processes include bonding, laminating, felting, packing, and/or other chemical and/or mechanical processes.

As defined herein, the term “textile” can be used to refer to any assembled network of yarns. Fabrics are textiles that are cut and organized into a specific shape for further processing (e.g., by sewing) into a finished garment, or other structure.

It is the spatial composition and interlacing pattern of yarns that are electrically conductive, electrically semi-conductive, and/or electrically non-conductive in the fabric that makes the yarns responsive to electrical, mechanical, chemical, and/or magnetic signals. For physiological measurements, it is advantageous to have fabrics that have a high electrical conductivity and are sensitive to specific ranges of, or changes within such ranges of, voltage, current, resistance, capacitance, and/or inductance, while such fabrics are substantially immune to noise that is voltage-, current-, resistance-, capacitance-, and/or inductance-based from the body being measured, the device itself, and/or the environment. Environmental noise can be classified as thermal noise, shot noise, partition noise, flicker noise, and/or burst noise and can be generated, for example, by nearby power lines, devices, and/or communication devices that emit radio frequency or electromagnetic radiation.

Woven fabrics are made of yarns interlaced in a horizontal and vertical binding system, also known as a weave. When constructing the weave, the vertical traveling (e.g., vertically-extending) threads are called the warp, and the horizontal traveling (e.g., horizontally-extending) threads are called the weft. The sequence of overlaying and underlaying the warp and weft create a woven pattern with specific characteristics. The three most common types of weaves are plain, twill, and satin weaves. A plain weave is formed when there is an equal number of overlays and underlays of warp and weft yarns, respectively, also known as creating a balanced weave. Twill and satin weaves have increasing ratios of warp to weft yarns, such as 1:2, 1:3, and so on, such that the warp yarns are exposed in a specific pattern on the top and bottom of the textile. There exist more complex weave patterns, in which the warp and weft yarns do not have repeated patterns and in which the yarns can change in direction to give the textile a non-linear pattern of warp exposure. Examples of complex weaves can include multiple plane, pile, inlaid, Jacquard, dobby, gauze, and/or leno weaves. When the weave is completed, the end of the threads at the edge of the fabric is woven with a high density of weft yarns to form a selvedge edge.

Parameters that impact a weave's mechanical and aesthetic properties can include, for example, the ends-per-inch (EPI), which is the number of warps per inch, and the picks-per-inch (PPI), which is the number of wefts per inch. In addition, the weight (e.g., density, denier, etc.) of the warp or weft yarn can also be selected to also contribute to the mechanical and/or aesthetic properties of the resultant textile. Both the EPI, PPI, and weight of the warp yarn generally determine the weight ratio of the textile. The weight ratio is the ratio of warp yarn to weft yarn and can be calculated by the number of yarns per inch, or the weight of the yarns per inch.

Woven fabrics are made from a weaving process of two components, a warp and weft yarn. These yarns are often perpendicular, crossing at approximately 90° angles; however, the construction process is not limited to one particular angle, and weaving can be done at any suitable angle between the warp and weft yarn. When viewing the fabric, the warp yarn extends in a generally vertical direction and the weft yarn extends in a generally lateral direction. EPI is the amount of warp yarn woven per inch of fabric. EPI can be selected to control a variety of mechanical characteristics of the fabric, such as weight, flexibility, stretchability, appearance, texture, and/or overall cost and labor that is required to weave multiple yarns at a specific density. A balanced weave is produced when there are the same quantity of weft yarns per inch as there are warp yarns per inch. Related to EPI is the weight ratio, which is the ratio of warp to fill, expressed as the percentage of warp yarn per inch. Factors that can be modified to control the weight ratio are, for example, the thickness and pliability of the yarn. Although two fabrics may have the same EPI, such fabrics may nevertheless have different weight ratios, as one may use a thicker yarn than the other.

Weave patterns are defined by how the warp yarn passes over or under the weft yarn. Although not limited to these examples, three basic weave types are disclosed herein as merely examples, and the subject matter disclosed herein is not limited in any way to only such weave types.

In plain weaves, the warp and weft yarns alternate in over and under passes with respect to the other in equal amounts, or instances. The warp and weft yarns can be arranged densely and the fabric can be relatively dense and thick.

Twill weaves have diagonal ribs, in which the warp yarn or the weft yarn passes in consistent multiples (e.g., two or more), such that the top yarn (e.g., the warp yarn) is intermittently exposed on the surface of the fabric. When multiple warp yarns are assembled, the structure looks as though the warp yarn is passing diagonally on the fabric. The fabric surface has obvious oblique lines, feel, luster and elasticity. The fabric has higher elasticity, and the weight of the warp and the weft fabric can be dissimilar to give the fabric a different feel and aesthetic in either direction. Typical applications for such twill weaves are in bedding.

Satin weaves are distinguished from twill weaves by the fact that satin weaves have fewer intersections of warp and weft yarn. Satin weaves produce a fabric with a higher sheen, which is produced by exposing more warps than wefts. Due to fewer intersections, and the larger lines of exposed warp thread, the exposed warp threads are called floats. The strength of fabrics produced using satin weaves is lower than for fabrics produced using either of plain or twill weaves. Because there are less intersection points in satin weaves, the yarns can be denser and thicker; however, the overall cost is typically higher. The twist of the yarn can also be controlled or increased to give the floats more texture or aesthetic.

Other weave types, such as Sateen, Pile, Jacquard, and the like follow a similar concept and will be understood by persons of skill in the art.

Factors such as yarn selection, fabric thickness, thread count, weave pattern, fabric weight, and finishing affect the mechanical properties of the resultant fabric.

Knitted fabrics are formed by interlooping yarns into an intermesh of loops. Knitted fabrics can be made of one or more yarns. Loops running lengthwise are called wales, and those running crosswise are called courses. Knitting can either be done with one needle, also called bearded needles, or with two main needles. Knitting is analogous to stitching yarns, much like sewing, but using thicker yarns that can loop into 2D and 3D textile structures. The stitches determine the type of loops and patterns to form the interlocking mesh of yarns, which thereby control the aesthetic and mechanical properties of the fabric. Knitting machines can perform these complex stitches in a 2D array or in a circular knit. Types of knitting can include, for example and without limitation, weft knitting and/or warp knitting. Weft knitting incorporates yarns in the horizontal direction. Basic stitch types for weft knitting are plain or jersey, rib, and purl. Warp knitting incorporates yarns in the vertical direction. Basic stitch types for warp knitting are raschel or tricot knits. During the knitting process, warp knitting can also include non-knitted threads for producing color, density, and/or additional texture effects. Of the types of knits, the technique that stitches yarns into loops can be from knit, purl, missed, and/or tuck stitches.

Net, lace, braiding or plaiting are special types of interlaced yarn structures which strategically loop, weave, and combine yarns to form specific and stylized patterns. While woven and knitted fabrics typically have uniform patterns that repeat in the X- and Y-directions, fabrics formed using net, lace, braiding, and/or plaiting can have regular and irregular patterns that give the resultant fabric unique visuals, such as stylizing fabrics with logos, images, or frills. Although these processes are more manual, either by hand-crafting specific patterns or programming machines to include these patterns, the advantage is the additional control in disrupting a pattern to form a desired island or shape that may be required for a specific segment of the fabric or garment. Braiding or plaiting is another type of interlacing method that can use two or more yarns or strips to form flat or tubular fabric.

Nonwoven techniques are used to form fabrics that are not created through an interlacing method such as weaving, knitting, lacing, etc. Instead, such fabrics are chemically and/or mechanically bonded together via other suitable techniques. Although not limited thereto, examples of such nonwoven techniques are disclosed herein as merely examples, and the subject matter disclosed herein is not limited in any way to only such techniques.

One such example nonwoven technique is felting, in which yarns are interlocked through mechanically pushing and deliberately intersecting yarns, such that the friction of the mesh structure creates a continuous and sturdy piece of fabric. Another example is bonding, in which an adhesive is used to combine multiple types of fibers together. In bonding, the fibers can be of different types and sizes, such that the final fabric is a composite of the physical, material, and aesthetic properties of each of the fibers bonded together. Another example is laminating, in which fibers are bonded and coated with a strong polymer to flatten and create a seamless layer of fabric. Examples include laminating with polyurethane or other foams. This method allows introduction of coated, or powdered, materials to form composite fabrics but only on the surface of the fabrics.

Using any of the techniques disclosed herein, a textile that is electrically semi-conductive to electrically conductive can be formed for use as an electrode for electrical signal detection and transmission. The primary purpose for an electrode is to allow signals, such as voltage and current, to pass through the electrode with low attenuation, high bandwidth, and low noise susceptibility. Noise can be differential noise, such as multiple signals being transmitted (e.g., simultaneously) along the same communication path or line, or can be common noise, in which noise comes from a common source, such as a power line or RF interference. In both instances, these noise types can come internally from the electrode or system being measured, such as from the way the textiles are woven, which may introduce noise, or external noise which cannot be controlled from the environment. In any circumstance, the construction and engineering of an electrode is advantageously selected and controlled to reject any noise that is not part of the signal of interest.

Signals can travel in two major directions through a textile. The first direction is through the strands of the yarn in the axial direction, in which a signal from one end (e.g., a first end) of the textile is transmitted to the other end (e.g., a second end) of the textile. The second direction is through the bulk of the yarn in the transverse direction (e.g., in the direction of the thickness of the textile). There are several factors which impact the signal quality as it passes through an electrode, including (1) whether the yarn is made with an inner core or outer shell of electrically conductive fibers; (2) whether the yarns are twisted or further coated with electrically conductive material within the textile; (3) the density of the yarns within the textile, such as EPI, PPI, and yarn density of the interwoven yarns; (4) the weave, lace, knit, or non-woven pattern used in forming the textile; (5) whether there is a homogenous over and underlay of yarns made with cored electrically conductive material, or coated electrically conductive material, or electrically non-conductive material, or whether there are regions which heterogeneously combine layering of these different yarn types; and (6) whether the yarns are spun with mixtures of electrically conductive and electrically non-conductive yarns, such that signals have to travel, or pass, between electrically non-conductive regions or layers of the textile. This list of factors is not exhaustive and other factors have been found to exist.

These factors are primarily determined based on the conductivity of the yarn, the interlaced pattern of the yarn, and how different yarn types interface with one another within the textile. These factors contribute to how a signal travels within the electrode and whether or not the signal is impeded with regions of incompatible conductivity, regions that are resistive or electrically non-conductive, regions that are have different (e.g., more or less) levels of susceptibility to RF and/or EM interference, etc. Both differential and common noise can be reduced or rejected by designing specific electrically conductive, electrically semi-conductive, and/or electrically non-conductive yarn types that are interlaced in specific noise-rejecting interlaced patterns. Typical signal processing cutoffs are at minimum a reduction by half, or a reduction of −3 dB. In some embodiments, noise reduction can have a lower limit of about −3 dB and, in some instances, noise rejection at ratios of 1/10 to 1/100 (e.g., about −20 dB to about −40 dB) have been achieved according to the subject matter disclosed herein. The equation for ratio to dB is 20*log 10 (ratio)

An example of a design is to create physical symmetry in the underlay and overlay patterns of electrically conductive to electrically non-conductive yarns in the horizontal and vertical direction of the textile. The yarns can be further twisted to incorporate axial symmetry, which advantageously is suitable for use in eliminating common noise, specifically from external RF and EM interference. The reason for this enhanced resistance to common noise is that, as the noise travels through symmetric passing of under and over laced conductive yarns, the common noise in each yarn attenuates the common noise in the other yarn, such that the common noise in each yarn is eventually cancelled out as it passes through the electrode. Another example is selecting a pattern such that the signal paths of the conductive regions in the electrode move in the same direction, for example, the VCC (e.g., power line) and GND (e.g., ground or shunt) signals enter and exit the electrode through multiple electrically conductive or electrically semi-conductive yarns interlaced in the same direction; according to such an example embodiment, the differential noise is exposed to each other throughout the yarn, such that the differential signal is attenuated (e.g., cancelled out) as it travels through the electrode.

In some example embodiments, the electrodes have selective regions of electrically conductive material, so that signals travel a specific direction or route. One such example comprises modifying the weave or knit pattern, such that electrically conductive or electrically semi-conductive yarns can pass through, in an angled direction, within the textile, an example being a Jacquard stitch. Another example includes embroidering an additional sewn yarn that creates a pattern or a biased stitch that directionally influences the movement of a signal within the electrode; the embroidered pattern, as well as by controlling the tension of the yarns or threads, can embed electrically conductive yarn above or below the weave or knit pattern, which allows signals to jump between layers in the electrode, as well as to bypass or enter regions of electrically conductive to electrically non-conductive patterns to aid in rejection of differential noise or common noise. The embroidered pattern is also advantageous for introducing connections between neighboring electrodes, or to connectors configured to send signals to another device or CPU.

Other patterns can include creating networks of electrically conductive and electrically non-conductive textile regions, in which they are highly susceptible to resistive, capacitive, and/or inductive signals, such that noise can be trapped in such regions. Examples include noise that has extremely low or high frequencies, signals with distinct frequency patterns, and/or signals that have radio or electromagnetic behavior, which tend to gravitate towards such regions. Due to repeated patterns in a textile, the desired signal can travel to alternative electrically conductive to electrically semi-conductive yarns while leaving behind noise captured in such regions.

Another noise type is mechanical noise and motion artifacts. Physical movement can distort the yarn's patterns and alter the signal path, causing the signal to be distorted. Causes can be movement of the system, stretching, twisting, bending, and/or pressure applied on the electrode, and/or a loose contact between the system and the electrode. To reduce the effects of mechanical noise, electrodes may be assembled with flexible or stretchable patterns that protect against such mechanically-induced signal disruption. In terms of alterations to the yarn to provide such functionality, modifications can include bulking, twisting, and/or the use of stretchable fibers or filaments. In terms of alterations to the textile to provide such functionality, modifications can include using stretchable interlacing patterns that allow flexibility between the horizontally- and vertically-assembled yarns. In terms of alterations to the electrode(s) to provide such functionality, modifications can include cutting, folding, and/or stacking electrodes in the horizontal and/or vertical directions, such that such mechanical forces can dissipate across a larger surface area on the electrode.

When electrodes are customized beyond the textile level, they are referred to herein as assembled electrodes. Assembled electrodes may, for example, have unique shapes that conform to the 2D and 3D geometric features of the body being measured, have layers which allow signals to travel between multiple patterns of electrically conductive or electrically semi-conductive regions in the transverse direction, or axial direction, or be embroidered with conductive yarns to guide signals between areas inside and outside of the assembled electrode. In addition, the assembled electrode must have an impedance that is matched to, or substantially the same as, the body being measured by performance of the selected mode to allow for maximum power transfer. To match impedance, the assembled electrode can be further embroidered with a layer of material that has an electrical conductivity from conductive to non-conductive, and/or the textile itself can be interlaced with a specific ratio of yarns to match the impedance while still allowing a predefined signal flow.

To perform a measurement of a body using a specific modality, such as EKG, EMG, EOG, etc., a plurality of (e.g., at least two) electrodes are required to measure the voltage and current differential between the points on the skin where such electrodes are respectively attached. The quantity of electrodes, the placement of the electrodes, the size of the electrodes, and how the electrodes are connected to the CPU determine the electrode set for the measured modality. For example, performance of an EKG requires electrodes to be positioned around the heart, whereas performance of an EMG requires electrodes to be positioned around the muscle(s) of interest. If multiple modalities are measured at once, these electrode sets must be combined to compromise for the different placement, size, and number requirements for each modality. The unification of electrode set(s) that is capable of measuring multiple modalities is called an optimized electrode set.

There are internal and external noise sources, which can be categorized as differential or common mode noise. Noise sources can be resistive noise, capacitive noise, inductive noise, and/or RF and EM interference. For resistive noise, altering the area or length of the yarn and/or the stitch pattern or direction can limit the signal's susceptibility to resistive changes. For capacitive noise, decreasing the distance between the electrically conductive and the electrically non-conductive regions, as well as limiting the exposure of the electrically conductive material to the environment, can limit the signal's susceptibility to capacitive changes. For inductive noise, limiting the number of loops, or radial patterns of electrically conductive yarns and electrically non-conductive yarns can limit the signal's susceptibility to inductive noise. In addition, selecting yarn materials that are less susceptible to magnetic fields can also limit inductive noise.

In some embodiments, a separate electrically conductive or electrically non-conductive pattern or layer is embroidered or stitched on the electrode to act as a sacrificial antenna or reservoir for noise collection. This strategy is typically done for RF and EM shielding, in which this layer absorbs signal interference and protects the signals traveling beneath it.

In addition, signals must have the proper impedance match such that the signal power transfer is greatest between conductive and semi-conductive material within the yarn, textile, electrodes, or between adjacent or connected electrodes.

There are also considerations in eliminating mechanical noise, such as motion artifacts or distortions in the textile, which disturb the flow of signals due to bending, stretching, twisting, and/or pressure applied on the electrode. Electrodes can be nicked, cut, or folded to improve flexibility so that mechanical forces are dissipated across the electrode. Electrodes can also be stitched with additional layers, or embroidered patterns to secure regions while allowing other directions for movement. Finally, electrodes can be interlaced with redundant yarn patterns so that multiple regions are collecting data upon the occurrence of other regions being distorted or displaced from the specified measurement location.

Once signals are transferred to the CPU, there are also firmware and software level considerations to post-process signals and filter noise. Filtering techniques include low-pass filters, high-pass filters, bandpass filters, and/or notch filters. These filtering techniques are intended to selectively remove noise from a specific frequency band. In addition, signals can be deconvolved and transformed into the frequency domain to further select regions of unwanted signals. In order to perform successful filtering, signals must be sampled at twice the Nyquist frequency in order to successfully interpolate any signal loss. Noise to filter are baseline wander, powerline interference, other signal injection such as undesired EMG (muscle activity), RF and EM interference, and electrode motion artifacts that can arise when the body moves or the electrode is otherwise displaced from the specified location for performance of the measurement via the selected modality. Mechanical artifacts can be filtered through n-point moving average filters, time varying-low pass filters, gaussian impulse response filters, and/or adaptive filters using machine learning models, such as, for example and without limitation, least means squares, recursive least squares, neural nets, and/or random forests.

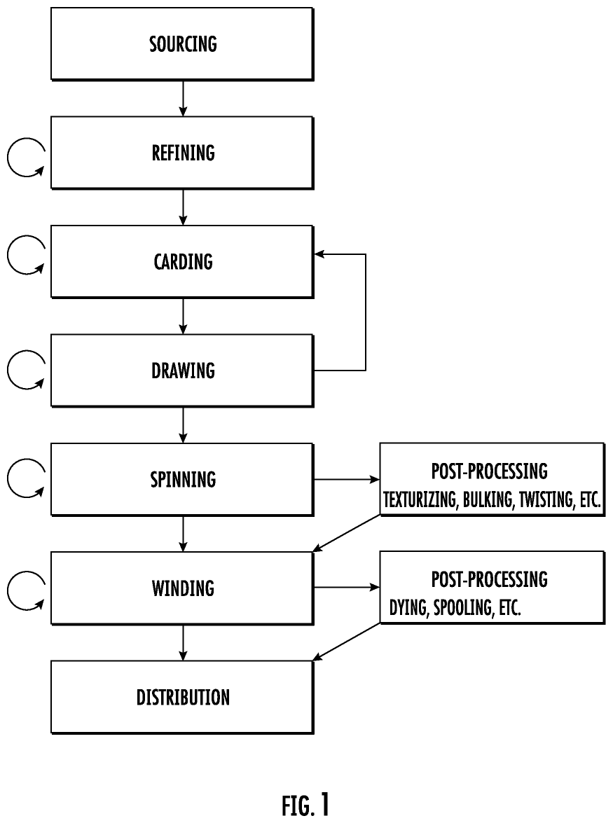

Referring now to the figures, example embodiments illustrating aspects of the subject matter disclosed herein are shown. FIG. 1 is a schematic illustration of steps for a method of making yarn, from the step of sourcing raw material for the yarn to the step of distributing finished yarn. As indicated in FIG. 1, one or more of the steps of the method can each be repeated multiple times depending on the manufacturer's requirements for the yarn to be produced. Repeating of a step may include repeating the step multiple times and can include the performance of any substeps, if applicable, any suitable number of times and in any combination or selection thereof.

The first step in the method is sourcing the raw material, which can come from natural sources (e.g., wool, cotton, silk, bamboo fiber, and/or any other suitable plant-based fiber) or synthetic manufacturing processes that produce, for example and without limitation, polyester, nylon, rayon, acrylic, spandex, kevlar, etc. The raw materials that are sourced can be in the form of pure batches or impure batches, in which case further refinement of such impure batches may be necessary based on the yarn being produced.

The next step in the method is refining the raw material, which can include sub-steps of cleaning, sorting, filtering defects, filtering based on size, weight, density, appearance, or adding chemicals or coatants in preparation for the performance of subsequent steps of the method. Another refining process is called teasing, which is used to loosen or unpack dense bundles of raw material so that the raw material can be more easily handled and managed in the subsequent steps of the method.

The next steps are carding and drawing, which can be performed iteratively, or in a looped manner. Carding is a mechanical process that cleans, further eliminates impurities in the raw material based on mechanical defects, disentangles, and/or mixes fibers within the raw material to produce a continuous film, web, and/or strand. Carding is advantageously performed to remove clumps and any mechanically deformed fibers that are unsuitable for being spooled or spun in subsequent steps of the method. Drawing, which can also be referred to as drafting, is used to further unbundle the fibers of the raw material into a looser assemblage. In the drawing step, mechanical force is applied to the fibers of the raw material to both stretch the fibers and draw the fibers in a specified orientation while simultaneously reducing the fiber strand size (e.g., diameter). Carding and drawing steps can be performed repeatedly until the fibers of the raw material are sufficiently aligned according to the manufacturer's requirements.

The next step in the method is spinning, which is an important step in forming a continuous strand of yarn. During the spinning step, fibers are twisted against one another to mechanically aggregate such fibers into a single strand. The fibers are continuously spun until they form a fixed diameter or weight and are then drawn out to form a continuous strand. Spun yarns can then optionally have one or more post-processing steps performed thereon, including mechanical processing steps of, for example, texturizing, bulking, and/or twisting the yarn to enhance its mechanical properties, aesthetic qualities, and/or texture.

After the completion of the spinning step (e.g., after the yarn is formed to a desirable or specified quality), the yarn a winding step can be performed, in which the yarn is wound into smaller units, such as spools or bobbins The winding step can, in some embodiments, be omitted. During and/or after the winding step, further post-processing steps, including dying of the yarn, can be performed on the yarn.

After the winding step is complete, a distribution step is performed, in which the finished yarns are distributed, for example, to the customers who have ordered such yarns.

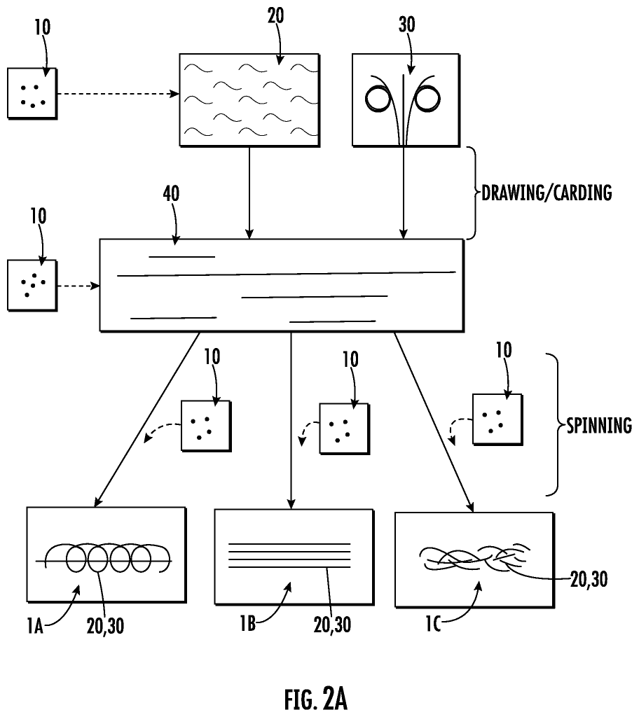

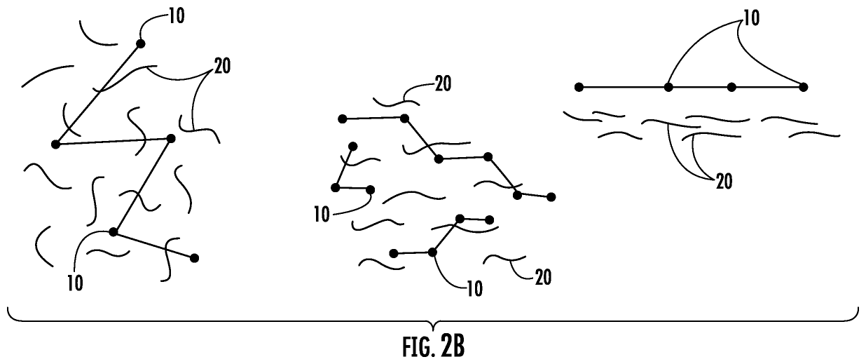

FIGS. 2A and 2B show the stages in which fibers are made, and at what process steps conductive material(s) 10 can be introduced into the fibers 20 to form an electrically semi-conductive or electrically conductive yarn. FIG. 2A shows the base materials that form a yarn, which can be formed via individual fibers 20 or filaments 30 made from an extrusion, pulling, polymeric, and/or chemical process. The fibers 20 or filaments 30 can, as shown in the top row of FIG. 1A, be made with conductive materials so that such fibers 20 and/or filaments 30 are inherently electrically conductive. Once these fibers 20 and/or filaments 30 are made, they can be mechanically processed through a drawing step and/or a carding step, which aligns individual monomers. During the drawing and/or carding steps, conductive material 10 can be added through deposition, annealing, electrical deposition, or a variety of chemical and coating process. Once fibers or filaments are aligned at 40, a spinning step is performed that mechanically or chemically oligomerizes the monomers of the fibers 20 or filaments 30 into yarns. During the spinning step, conductive materials 10 can once again be introduced, but at the yarn formation step through deposition (e.g. electroless, chemical, physical), coating, spraying, annealing, or a variety of chemical and coating processes. Types of yarns that can be assembled with conductive materials 10 are solid core yarns, generally designated 1A, with wound fibers 20 and/or filaments 30 around a solid core. In solid core yarns 1A, the core and/or the spun fibers can be electrically conductive. Another example type of yarn is standard filament yarn, generally designated 1B, which can be twisted or untwisted and in which the individual fibers 20 and/or filaments 30 and/or the coating over the filaments are electrically conductive. Another example type of yarn is spun yarn, generally designated 1C, in which fibers 20 and/or filaments 30 are twisted together to form a continuous chain of fibers 20. Such yarns can be bulked and/or further twisted into plys for higher density, texture, weight, and thickness.

FIG. 2B shows how the resistance, capacitance, inductance, voltage, and current behavior of a fiber 20 can be improved during such methods of manufacturing yarn. As shown in the left image of FIG. 2B, disorganized fibers 20 have a heterogeneous and discontinuous network of electrically conductive materials 10, which makes the resistance of the fiber 20 high, as the electrical signal does not have a straightforward path between two end-points. This also means that the ability to transfer electrical signals, such as in the form of voltage and current, is low. During the carding and drawing steps, the fibers 20 are better aligned, as shown in the center image in FIG. 2B, such that the average signal path between points in the network of electrically conductive materials 10 is reduced during the carding and drawing steps. With a reduced average signal path, the resistance of the bundled fibers 20 is reduced, allowing electrical signals to pass therethrough more efficiently. During the spinning step, the fibers 20 are further aligned and elongated in a single direction to increase the uniformity of electrical conductivity and also decrease the average path length, as shown in the right image of FIG. 2B. Spun yarns have greater ability for the transfer of electrical signals. Measurements using Ohm's law to calculate resistance, as well as Kirkoff's Laws to measure the average current and voltage signal change through the network of fibers 20, can be used to simulate how well such yarns can propagate a signal from one point to the next (e.g., along the length thereof) within the network or electrically conductive materials 10.

A similar assessment can be made with the capacitive and inductive characteristics of the fibers. Both the capacitance and inductance decrease as the fibers 20 become more aligned and densely packed, allowing signals to more easily travel between two endpoints of the network or electrically conductive materials 10 without being carried to other capacitive or inductive effects between fibers 20.

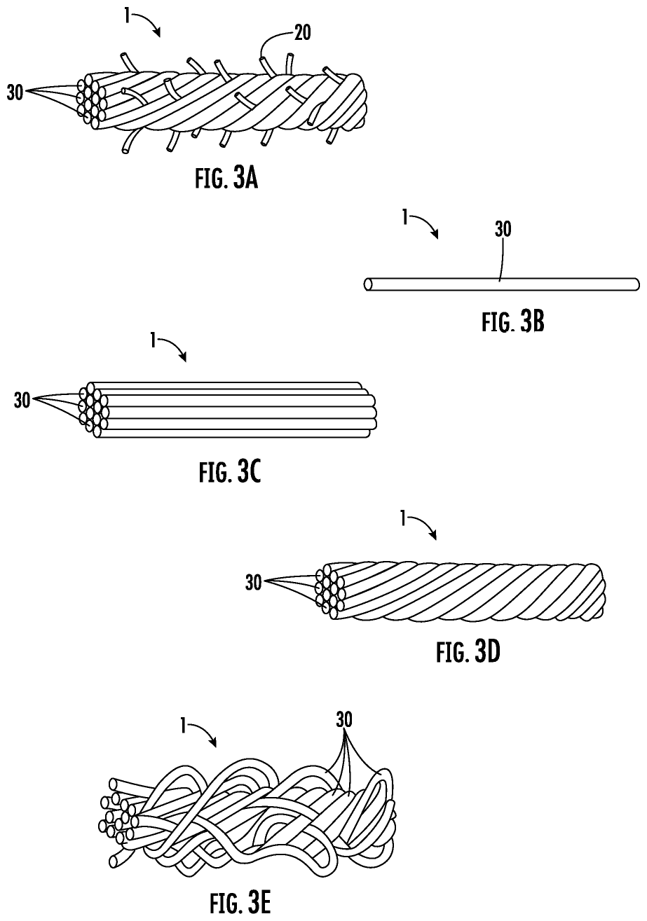

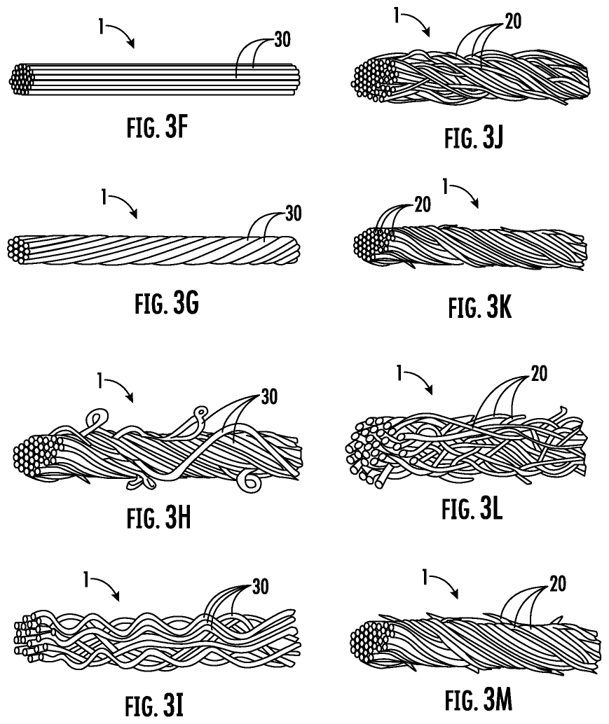

FIGS. 3A-3M shows the different types of yarns, generally designated 1, that can be formed using fibers 20 or filaments 30, and how such yarns 1 can be further modified through mechanical post-processing, such as twisting and bulking to enhance specific material features like texture and density. FIGS. 3A-3E each show a side-view of a traditional yarn 1 made from spinning fibers 20 or filaments 30 together. In FIG. 3A, a staple yarn, or spun yarn, comprising fibers 20 is shown. In FIG. 3B, a single filament 30 (e.g., a monofilament) is shown, which can itself define a yarn 1. In FIG. 3C, a yarn 1 comprising a plurality of untwisted filaments 30 is shown. In FIG. 3D, a yarn 1 comprising a plurality of twisted filaments 30 is shown. In FIG. 3E, a yarn 1 comprising a plurality of bulky filaments 30 (e.g., bulked filaments 30) is shown. These yarns 1 can fray, which the term used to describe the loosening of fibers 20 and/or filaments 30 and unraveling of the spun core. This typically happens after multiple uses, or when the yarn 1 experiences extreme mechanical stress when used. There also exists single filaments 30, shown in FIG. 3B, which if long enough with high tensile strength, can act as a yarn 1 itself. Examples of single-filament yarns 1 are spandex or polyester which have a low young's modulus yet high tensile, durability, pliability and malleability and are resistant to tears and stretch. Multiple filaments 30 can be combined to form a yarn 1 with a core of tightly bound filaments 30, as shown in FIG. 3C. This bundle of filaments 30 is also known as untwisted filament yarn 1. As shown in FIG. 3D, the filaments 30 of the untwisted filament yarn 1 can be twisted to further secure the bundle of filaments 30 together to create the twisted filament yarn 1 shown in FIG. 3D. After twisting, either spun yarn 1 comprising filaments 30 and/or fibers 20 can be bulked, the result of which is shown in FIG. 3E. Bulking is when air is introduced into the yarn 1 which separates and loosens the twisted fibers 20 and/or filaments 30. This allows for more pliability of the yarn 1 which may be required for textiles that need to be more flexible or agile on the worn surface. In addition, bulking is used to change or enhance texture, density, and aesthetics of the yarn 1.

FIGS. 3F-3M each show different variations of yarn 1 when using spun fibers 20 or filaments 30. The examples shown in FIGS. 3F-3I each show an example embodiment of a yarn 1 made from one or more filaments 30. In FIG. 3F, the yarn 1 comprises untwisted filaments 30. In FIG. 3G, the yarn 1 comprises twisted filaments 30. In FIG. 3H, the yarn 1 comprises bulked twisted filaments 30. In FIG. 3I, the yarn 1 is comprises filaments 30 that are gimped, looped, or wrapped beforehand to create a twisted and wound-up filament 30. These filaments 30 filaments are then spun to form a stretchable yarn 1. Alternatively, spun filaments 30 can be bulked and gimped in yarn form to create a stretchable yarn 1.

The examples shown in FIGS. 3J-3M each show an example embodiment in which fibers 20 are processed to form different textured yarns 1 with varying mechanical strengths. In FIG. 3J, the fibers 20 are only carded before execution of a spinning step; in this example, fibers 20 are looser and less organized, creating a lower quality yarn 1 with a rougher texture. In FIG. 3K, a yarn 1 is shown, which is made from fibers 20 that have been processed in a carding step, a drawing step, and a combing step, which better aligns the fibers 20 and allows a better spun yarn 1 with enhanced texture and mechanical performance. In FIG. 3L, woolen yarn 1 is shown, which is made from fibers 20 that are typically derived from wool or other animal-based materials that have shorter fibers 20 that vary in diameter and length; such fibers 20 are more heterogeneous, so they typically still have unorganized fibers 20 packed after completion of the spinning step; woolen yarn 1 is typically only processed via a carding step. In FIG. 3M, worsted yarn 1 is shown, which is yarn 1 that is of higher quality than woolen yarn because such yarn 1 is formed via fibers 20 undergoing a carding step, a drawing step, a combing step, and a twisting step, in which the fibers 20 are highly twisted, to produce a medium weight yarn 1 with enhanced mechanical properties and physical texture.

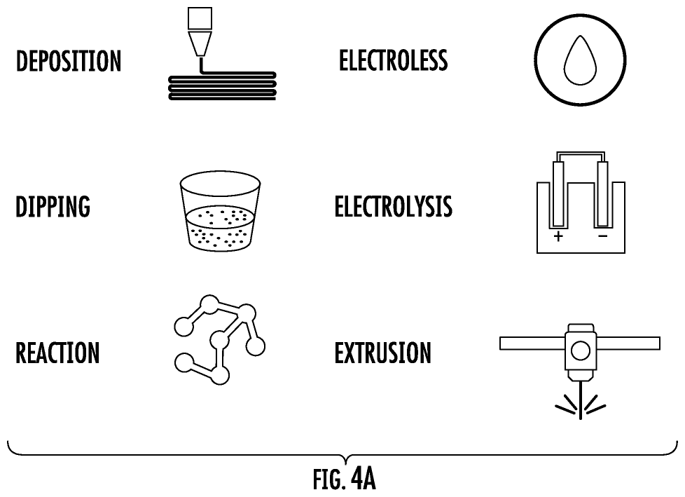

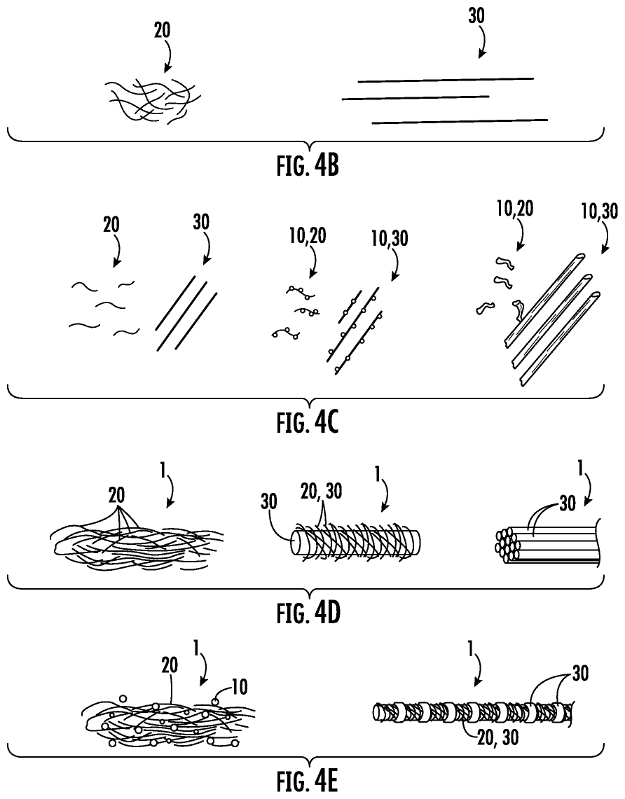

FIGS. 4A-4E show examples of different options and combinations of materials that can comprise an electrically conductive yarn 1. The options shown in FIGS. 4A-4E can be used in any quantity and combination to create a unique yarn type. The first option, shown in FIG. 4A, is the type of coating or synthesis process to incorporate conductive material into a fiber 20 or filament 30. Examples are deposition methods, such as chemical vapor, physical, electroless deposition, dipping, extrusion, electrolysis, and/or other electrical, mechanical, chemical, and/or magnetic processes to combine conductive material when generating the fiber 20 or filament 30. Another option, shown in FIG. 4B, is whether a fiber 20, filament 30, or a combination of fiber(s) 20 and filament(s) 30 is used when spinning the yarn to be produced. Another option, shown in FIG. 4C, is which, if any, further processing steps will be implemented for the fiber 20 or yarn, as the case may be, to form coating(s) of electrically conductive material thereon. Another option, shown in FIG. 4D, is the type of spinning process used to create a yarn 1. In the left image of FIG. 4D, a spinning process is used that spins smaller fibers 20 into a continuous chain. In the center image of FIG. 4D, a core 50 is used in the spinning process; the core 50 can be made from a single, multi-filament 30, pre-spun, and/or packed fiber 20 collection, which is then wrapped (e.g., around the outer surface thereof) in spun fibers 20. In the right-most image of FIG. 4D, multiple filaments 30 or assembled fibers 20 are packed into a single core yarn 1. Each yarn 1 can be further processed by bulking, elongating, and/or twisting to improve texture, density, and/or mechanical properties of the yarn 1. As shown in FIG. 4E, after such yarns 1 are formed, a conductive layering process may be performed on such yarns 1; one example of such conductive layering process is by coating, while another is to use a conductive filament 30 or assembly of fibers 20 and twisting it around the original yarn 1.

For the example embodiment shown in FIGS. 4A-4E, there are at least two options for raw material selection, fiber 20 and/or filaments 30; at least three main options for conductive processing of fibers 20 and filaments 30; at least three options for yarn-making, using conductive, semi-conductive, and non-conductive fibers 20 and filaments 30; and at least two options for coating an assembled yarn 1 with electrically conductive polymers and/or materials. Thus, even omitting the conductive processing steps shown in FIG. 4A, which may contain a multitude of options, at a minimum there exist at least 36 unique combinations of steps to create an electrically conductive yarn 1 when one process per step is selected. The total number of unique combinations increases exponentially if mixing and adding additional steps to each process is considered, such as using both fibers 20 and filaments 30 in the yarn 1, introducing new conductive coating methods, and/or using alternative yarn making processes.

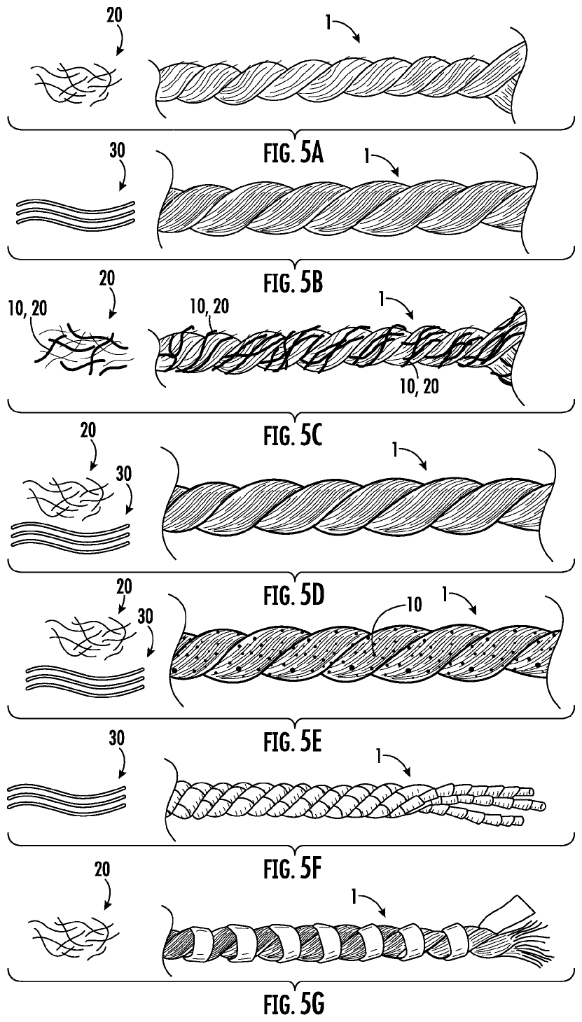

FIGS. 5A-5G show further example embodiments of yarns, generally designated 1, that can be produced in the spinning step, such that yarns 1 that is electrically conductive, semi-conductive, or non-conductive can be made using different combinations of electrically conductive, semi-conductive, and/or non-conductive fibers 20 and/or filaments 30. FIG. 5A shows a yarn 1 produced via a traditional spinning technique on/with fibers 20. FIG. 5B shows a yarn 1 produced via a traditional spinning technique on/with filaments 30. FIG. 5C shows a yarn 1 that is similar to the yarn 1 shown in FIG. 5A but incorporates a mixture of electrically conductive fibers 20 (designated using solid lines) and electrically non-conductive fibers 20 (designated using broken lines) to form a yarn 1 with an electrical conductivity that can range from electrically conductive to electrically semi-conductive. Another example that is substantially similar to that which is shown in FIG. 5C uses a mixture of electrically conductive filaments 30 and electrically non-conductive filaments 30 to form a yarn 1 with an electrical conductivity that can range from electrically conductive to electrically semi-conductive, and which would appear substantially similar to the yarn 1 shown in FIG. 5B. FIG. 5D shows a yarn 1 made from a combination of electrically conductive and electrically non-conductive fibers 20 and filaments 30. In FIG. 5D, the fibers 20 of the yarn 1 are spun (e.g., twisted) around a core made of one or more filaments 30; the filament(s) 30 can be electrically conductive and/or electrically non-conductive and the fiber(s) 20 can be electrically conductive and/or electrically non-conductive. FIG. 5E shows a yarn 1 made from electrically non-conductive fibers 20 and filaments 30, which are then coated with electrically conductive material 10, which is indicated by the dots illustrated in FIG. 5E; the electrically conductive material 10 can be added to the fibers 20 or filaments 30 before spinning and/or the electrically conductive material 10 can be added to the spun yarn 1 afterwards. FIG. 5F shows an example embodiment of yarn 1 in which filaments 30 are entirely coated (or wrapped, also known as a sheath) in an additional layer of electrically conductive material 10; these coated filaments 30 are then spun into the yarn 1 shown in FIG. 5F. FIG. 5G shows a yarn 1 made from electrically non-conductive fibers 20, which is afterwards (e.g., in the manner of post-processing of the yarn 1) additionally twisted with a layer of electrically conductive fibers 20 and/or filaments 30.

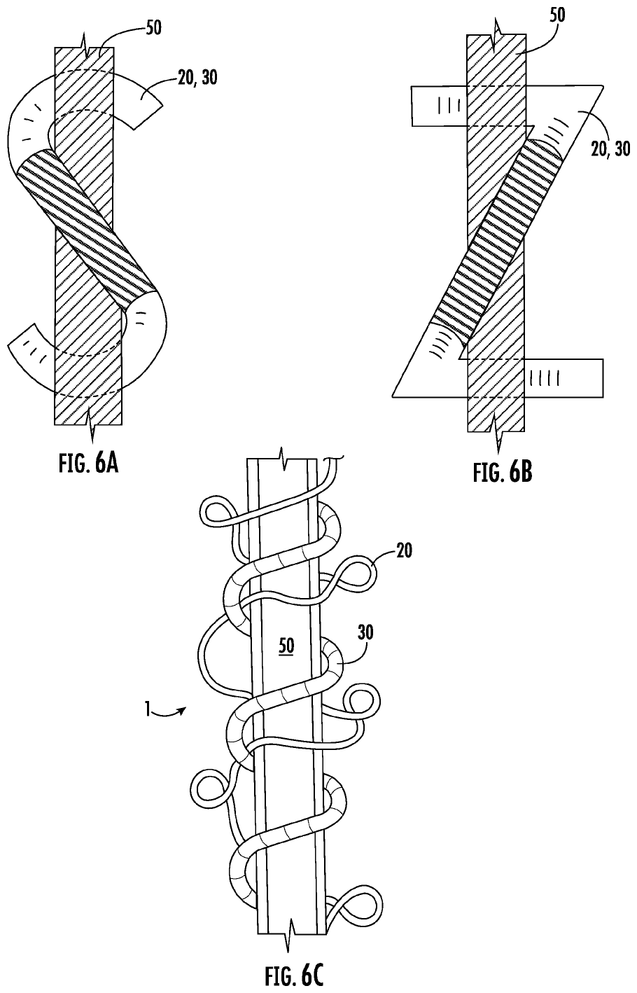

FIGS. 6A-6C show example embodiments regarding the effect of the direction, rate of twisting, and ratio of twisted fibers 20 or filaments 30 around each other or a core 50 can imbue the resultant yarn formed with advantageous mechanical and/or electromechanical properties. FIGS. 6A and 6B show the different directions a fiber 20 or filament 30 can be twisted around a core 50. As shown in FIG. 6A, an “S” twist produced when the fiber 20 or filament 30 is twisted around the core 50 in the clockwise direction, relative to the axial direction of the core 50. As shown in FIG. 6B, a “Z” twist is produced when the fiber 20 or filament 30 is twisted around the core 50 in the anticlockwise direction, relative to the axial direction of the core 50. Both of the twist directions shown in FIGS. 6A and 6B can be implemented in a single yarn with the purpose of controlling which twisted layer(s) of material is exposed and which twisted layer(s) is hidden beneath the following twisted layer. In addition, different twist patterns can confer different textural and aesthetic feeling and appearances, which influence how the final yarn is worn on the body. FIG. 6C is an example illustration of how precisely controlling the direction of twist, as well as the tension and compactness of the twisted layers, can be used to produce a unique yarn type. In FIG. 6C, a core 50 is twisted with fibers 20 and filaments 30, known as effects and binders, on the surface of the core 50. The term “effect” is used to refer to stylized fiber(s) 20 and filament(s) 30 that introduce other electrical, mechanical, chemical, and/or magnetic properties to the yarn, generally designated 1. Such effects can have, for example and without limitation, different elasticity, different electrically conductivity, different magnetic properties, different chemically reactivity, and the like, in comparison to the material from which the core 50 is made. Such effects are twisted with a binder layer to hold the effect layer onto the core 50 in the prescribed location. The density and twist tension between the binder layer and the effect layer can be used to control the exposure of the effect layer, in addition to controlling the texture of the yarn 1 at specific segments around the core 50. This control is advantageous for allowing certain segments, or frequency of segments, of a yarn 1 to exhibit one or more different properties from the core 50. An example of such an application is controlling for conductive regions along the length of the core 50, which may be desired if specific conductive patterns are required to interface with neighboring materials, such as for transferring signals at a desired rate and quality.

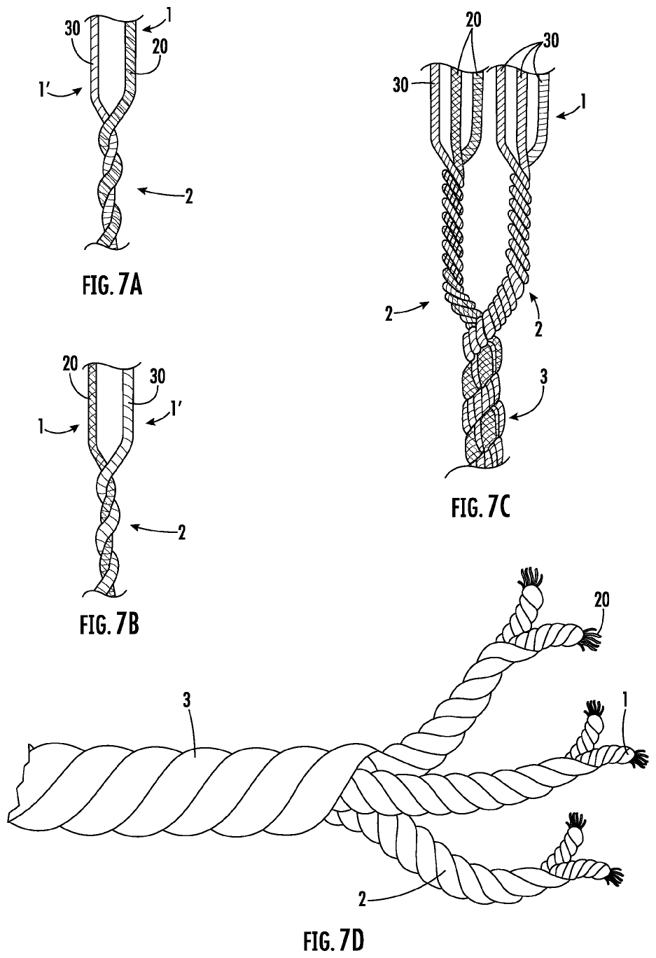

FIGS. 7A-7D show examples of how twisting fibers 20, filaments 30, and/or yarns, generally designated 1, can be used to create secondary strand structures. FIGS. 7A and 7B shows how a yarn 1 can be formed by twisting a fiber 20 and a filament 30 in the same direction (FIG. 7A) or opposite direction (FIG. 7B) as a secondary yarn, generally designated 1′, to form a ply yarn, generally designated 2, (e.g., a two-ply yarn) with ZZ twist, ZS/SZ twist, or SS twist. As shown in FIG. 7C, such ply yarns 2 can be twisted with other ply yarns 2 in Z and/or S twist combinations to form cords 3, which can also be referred to as cables. Such cords 3 can be further twisted in Z and/or S twist combinations to form ropes. Ropes, and sometimes cords 3, are more specialized yarns with heavier weights for more rugged applications, such as towing, wrapping, and/or shielding materials, especially for larger bodies, such as vehicles, furniture, and/or infrastructure. FIG. 7D is a detailed view of a strand structure, in which the different instances of fibers 20, yarns 1, ply yarns 2, and cords 3 are shown. Each unit within the strand structure can be made of, or doped with, electrically conductive material, such that the entire strand structure can be provided with an electrical conductivity within the range of electrically conductive to semi-conductive. Furthermore, any and/or all of the units of the strand structure can be twisted and combined with effects and binders that have respective electrical conductivities ranging from electrically conductive to electrically non-conductive to produce additional unique yarn types.

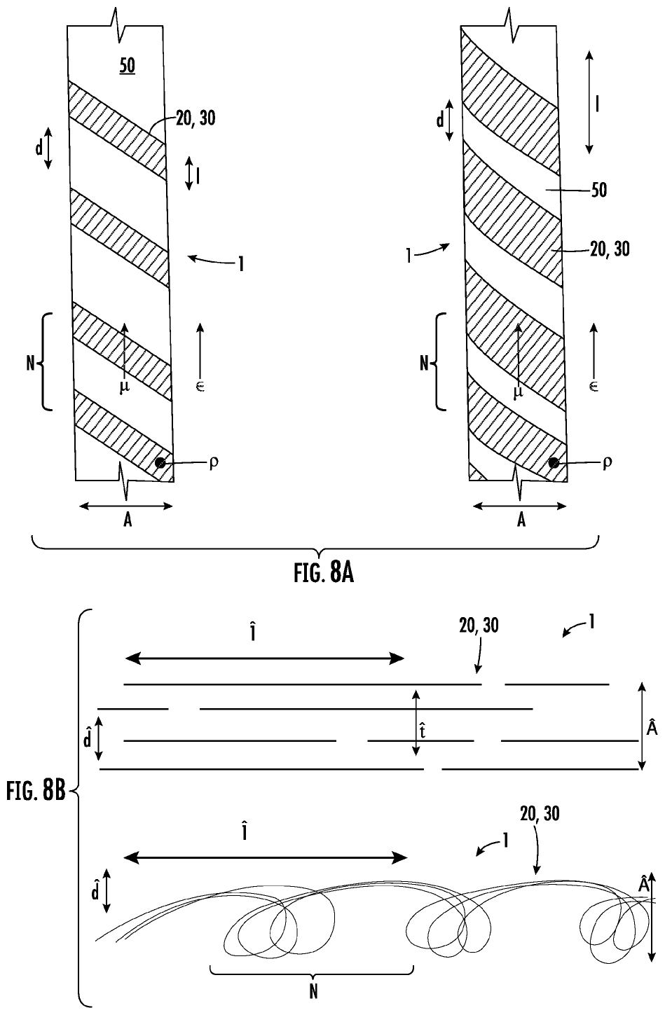

FIG. 8A shows example embodiments of a yarn, generally designated 1, with a core 50, which can be electrically conductive, semi-conductive, or non-conductive, that is twisted with a material (e.g., fiber(s) 20 and/or filament(s) 30) that is electrically conductive, semi-conductive, or non-conductive. In the left image of FIG. 8A, a first example embodiment of a yarn 1 is shown, in which the yarn 1 comprises an electrically non-conductive core 50 twisted with an assembly of electrically conductive fibers 20 and/or filaments 30. In the right image of FIG. 8A, a second example embodiment of a yarn 1 is shown, in which the yarn 1 comprises an electrically conductive core 50 twisted with an assembly of electrically non-conductive fibers 20 and/or filaments 30. In both of the example embodiments shown in FIG. 8A, the yarn 1 is designed to control the exposure of the electrically conductive material, which determines the electrical properties of the respective yarns 1. Such electrical properties can include, for example, resistance (inversely known as conductance), capacitance, and inductance. Such electrical properties are, in some embodiments, directionally specific, which are the axial and transverse direction of the yarn 1 with respect to the core 50. Intrinsic parameters of the yarn 1, as well as the density of the twisting (e.g., the number of twists per length) and the thickness of the twisted material can be selected to control the resistivity (ρ) of the material from which the yarn 1 is made, electric and dielectric permeability of the material from which the yarn 1 is made (referred to as electric constant hereout, ε, κ), and permeability (μ) of the material from which the yarn 1 is made. The resistivity is correlated to the material's resistance according to the following equation:

$$R = ρ \frac{L}{A}$$

The electric constant is correlated to the material's susceptibility to capacitance according to the following equations:

$$C = ϵ \frac{A}{d} or$$

$$C = ϵ \cdot κ \frac{A}{d}$$

The permeability is correlated to the material's susceptibility to inductance according to the following equation:

$$L = μ \frac{{N}^{2} \cdot A}{l}$$

The resistance and capacitance values of the yarn 1 can be observed (e.g., measured) in the axial and transverse directions of the yarn 1. The inductance value of the yarn 1 can only be observed in the axial direction, perpendicular to the twisting direction, unless the yarn 1 itself is twisted in the transverse direction (e.g., with another yarn) to form a ply yarn (e.g., 2, see FIGS. 7A-7D), cord (e.g., 3, see FIGS. 7C, 7D), or rope, which exposes magnetically susceptible loops (N).

The resistivity of the yarn 1 is determined by whether or not the yarn 1 is made with an electrically conductive or non-conductive core. A conductive core has a larger area A, thereby which causes a decrease in the resistivity. A non-conductive core with a twisted conductive layer typically has a higher resistivity, since the area is reduced and, additionally, the traveled distance increases because of the additional distance, or length, caused by the winding that occurs around the core. However, altering the material type, such as exchanging between a copper or silver core or twisted material also impacts the resistivity p of the overall yarn 1. In the transverse direction, it is the ratio of exposed conductive material to the hidden conductive material caused by the twists. The distance in the axial direction (e.g., the width, measured in the axial direction) of exposed non-conductive material, or the distance between directly adjacent twists of the electrically conductive material, is denoted as d. The distance in the axial direction (e.g., the width, measured in the axial direction) of exposed electrically conductive material is denoted as l. An increase in the ratio of d to l reduces the resistivity of the material in the transverse direction. Correspondingly, a decrease in the ratio of d to l increases the resistivity of the material in the transverse direction. The thickness and density of the twisted material can be selected to control the ratio of d to l for a yarn 1. In the first example of FIG. 8A, in which the core is electrically non-conductive, choosing a thinner and less dense twist of electrically conductive material increases the resistance of the yarn 1. Increasing the covered area of the electrically non-conductive twist material and/or increasing the twist density, or the number of twists per unit length (N), around an electrically conductive core decreases the electrical resistance of the yarn 1. Conversely, decreasing the covered area of the electrically non-conductive twist material and/or decreasing the twist density, or the number of twists per unit length (N), around an electrically conductive core increases the electrical resistance of the yarn 1.

The susceptibility to capacitance of the yarn 1 can be determined in a similar manner. For a yarn 1 with an electrically non-conductive core, increasing the distance (l) of the exposed electrically conductive material relative to the distance (d) between directly adjacent twists of the electrically conductive material increases the susceptibility to capacitance of the yarn 1. In the axial direction, capacitance is formed between each twist segment (e.g., length of material around a full rotation of 360° about the core). Therefore, increasing the distance/relative to the distance d between each adjacent twist segments increases internal capacitance susceptibility of the yarn 1. Also, an increase of the area A of the yarn 1 will increase the internal capacitance susceptibility of the yarn 1. A yarn 1 with an electrically conductive core does not have significant (e.g., only negligible, or no) internal capacitance. However, this is not the case when capacitance with external objects, such as a neighboring yarn in the transverse direction, is considered; in such a case, the exposed conductive material is now the interfacing area A of the yarn 1. The amount of exposed electrically conductive material, whether in the form of a twisted electrically conductive material or from exposed patches of an electrically conductive core, determined the surface area in which capacitance can be felt. Any layers between the electrically conductive material contribute to the dielectric layer K. The distance between neighboring yarns, or twist segments, determines d, so mechanical changes to the yarn 1, such as are induced in the carding, drawing, compacting, spinning, twisting, and/or bulking steps, can be used to change the distance d to a specified, or preferred, value.

The inductance of a single yarn 1 is most significant in the axial direction for an electrically conductive material twisted along an electrically non-conductive core. The twist density (N) around the core is directly proportional to the inductance of the resultant yarn 1. Other parameters of interest in determining inductance of the yarn 1 include the area of the yarn 1 (e.g., the thickness or weight of the yarn) and the length of the yarn 1. The permeability u of the yarn 1 is intrinsic to the material type of the core and the material used for twisting around the core. Selection of materials for the core and twists that have high values for electrical conductivity and magnetic activity will cause the permeability constant to be high and, thus, the inductance thereof will also be high. Stated somewhat differently, the permeability constant and inductance of the yarn 1 are proportional to the electrical conductivity and magnetic activity of the materials used in forming the yarn 1. A yarn 1 formed with an electrically conductive core will have low inductance when the material that is twisted around the core is electrically non-conductive and, therefore, the yarn 1 formed will have little to no permeability; however, when such a yarn 1 is twisted along a base yarn 1 (e.g., an electrically non-conductive yarn 1) to form a ply or cord, the density of twists of the electrically conductive yarn 1 to the base yarn 1 determines the twist density N and the weight and thickness of the electrically conductive yarn 1 contributes to the permeability u of the ply yarn or cord. The thickness and length of the base yarn 1 determines the values of both A and I.

FIG. 8B shows two example embodiments in which, respectively, the use of straight or twisted fibers 20 and/or filaments 30 can be selected to affect and/or control the resistance, capacitance, and/or inductance of the yarn 1 at the level of the fibers 20 and/or filaments 30. There is an average length an electron has to travel within the yarn 1, denoted by \hat{l}, which is the average length of continuous fibers 20 and/or filaments 30 that extend within the yarn 1 that a signal can follow before having to “jump” to, or be transmitted into, another fiber 20 and/or filament 30, as the case may be. In addition, there is an average distance between fibers 20 and/or filaments 30, denoted by \hat{d}. The area A is defined as the total thickness, or width (e.g., in the manner of a diameter) of the yarn 1. These parameters, along with the chemical composition of the fibers 20 and/or filaments 30 determine the electrical resistance of the yarn 1. The yarn 1 also exhibits an electrical resistance in the transverse direction, which is dictated by the average transverse distance an electron has to travel, denoted as \hat{t}. Both \hat{l} and \hat{t}, when summed across the entire length and thickness of the yarn 1, are greater than the length and thickness of the yarn 1.

The yarn 1 also has an internal capacitance, which is the capacitance experienced per fiber 20 and/or filament 30 in the entire bundle. The internal capacitance is determined by the exposed surface area per continuous fiber 20 and/or filament 30, and the distance \hat{d} between the strands of fiber 20 and/or filament 30. The external capacitance, which is the capacitive effect when placed against (e.g., so as to be in direct contact with) another conductive body is dictated by the shape of the yarn 1 and the distance of the yarn 1 from a neighboring conductive material. The parameter that changes because of \hat{l}, \hat{d}, and \hat{t} is the electric constant and dielectric constant ε and κ, respectively, which defines the intrinsic ability of the yarn 1 to store charge within the packed fibers 20 and filaments 30.

For twisted yarns 1, \hat{l}, \hat{d}, and \hat{t} are smaller than for untwisted yarns, meaning the axial and transverse resistance values decrease. Although the internal capacitance of the yarn 1 should otherwise increase as a function of a decrease in \hat{d}, twisting of the yarn(s) 1 actually reduces or eliminates a capacitive effect, since the fibers are in perfect contact with their neighbors allowing full transmission of signal between fibers making them act as conductors.

There is no intrinsic inductance for an untwisted yarn 1 as there are few or negligible looped areas within the fibers 20 or filaments 30 of such untwisted yarns 1. However, twisted yarns 1 create loops for the electrical signal to produce a magnetic field when voltage and current is introduced. Inductance is proportional to the twist density N. Additionally, the permeability K is determined by the core the yarn 1 is twisted on, around, and/or about, the chemical composition of the fibers 20 and/or filaments 30 of the yarn 1, and the uniformity of the fibers 20 and/or filaments 30 of the yarn 1, which are correlated to \hat{l}, \hat{d}, \hat{t}, and A.

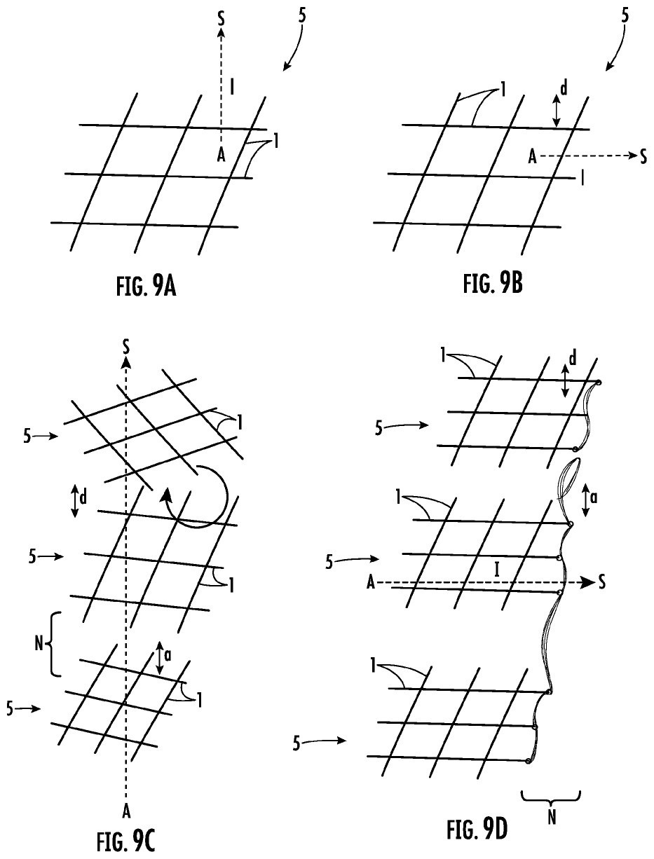

FIGS. 9A-9D show how signals travel through the axial or transverse direction of a textile 5 made of horizontal and vertical interlacing of conductive, semi-conductive, and non-conductive yarns 1. Textile 5 is used to describe any interlace of yarns 1 in the X, Y, or Z direction constructed from a woven, knitted, laced, or non-woven method. FIG. 9 focuses on signals traveling through a textile 5 that is woven, but a similar analysis can be done with knitted, laced, or non-woven methods. A cut, folded, sewn, embroidered, stacked, or other engineered shape of a textile 5 tailored for electrical signal pickup is referred to as an electrode.

FIGS. 9A and 9B show, respectively, how a signal can travel through (transverse), or within (axial) a textile 5 given a conductive arrangement of interlaced fibers, filaments, and/or yarns 1. Such textiles 5 are advantageously designed to preferentially allow signals of interest (e.g., from modalities including, without limitation, EKG, EMG, EOG, EEG, BIA, EDA, and SIA) to be transmitted through the electrode with minimal signal loss and/or noise injection. The signals are captured from the surface of the system, travel through a network of yarns (e.g., electrically conductive yarns) in a textile 5 constructed as an electrode, and interface with an electrical connection to a CPU, which is used to process the signal and transfer the data obtained from the signal for analytical purposes. In addition, the textile 5 is particularly advantageously mechanically tolerant to motions, such as bending, stretching, twisting, and/or pressure, so that the signal is not distorted during transmission due to such mechanical deformations in the interlaced yarns. The density of laced yarns, as well as the pattern in which they are arranged in the horizontal and vertical direction, can be used to provide signal selectivity and/or noise filtering functionality. Noise can be classified as differential noise or common mode noise. Textiles 5 that are twisted, woven, laced, and/or knitted in a repeating pattern can advantageously be designed to reject common noise. Textiles 5 that allow signals to enter multiple points and which transmit signals in parallel directions can advantageously be designed to reject differential noise. Furthermore, signals that travel between alternating paths of electrically conductive or semi-conductive yarns around electrically non-conductive yarns can be used eliminate or attenuate noise as the signal travels through the electrode. Additionally, gaps, or patterns of electrically conductive and/or semi-conductive regions surrounded by electrically non-conductive regions, or vice-versa, can be used to create a guided signal path within the electrode to control how signal and noise is attenuated or filtered upon entry and exit of an electrode. In addition, cut, folds, and/or layering of electrically conductive, semi-conductive, and/or non-conductive yarns can effectively act as reservoirs that absorb unwanted signals while allowing the desired signal to be transmitted on another yarn layer.

The textile 5 can be further engineered with structured electrically conductive, semi-conductive, and/or non-conductive yarn patterns to minimize susceptibility of the textile 5 to parasitic capacitance or inductance.

Advantageous patterns are used to eliminate distances between unwanted conductive material d, reduce the number of unwanted loops of conductive material N, alter the mean travel path of the signal l, and/or alter the conductive area A a signal travels through by modifying the density of horizontally and/or vertically laced yarns. Another consideration in designing such textiles 5 is to design yarn patterns that are immune or shielded from radio frequency (RF) and/or electromagnetic (EM) interference. RF and EM signals can come from powerlines, other devices, or bodies that are electrically active. Shielding functionality can be provided by creating a “sacrificial” layer of electrically conductive yarn that is dedicated to absorption of electromagnetic interference, thereby protecting the yarn within an embedded or interlaced layer within the textile 5, through which the signal is transmitted. Ideal shielding requires covering the yarns carrying the signal through a barrier of sacrificial electrically conductive material on the surface. Therefore, overlaying the signal-transmission yarn layer(s) with an electrically non-conductive material, between the sacrificial layer and the signal-transmission layer(s), to pad the sacrificial and signal-transmission layers to prevent signal leakage therebetween can be achieved through weaving, knitting, and/or lacing methods of yarn-making. This padding effect can also be achieved by cutting, folding, embroidering, and/or stacking textile-based electrodes in the horizontal and/or vertical direction.

FIGS. 9C and 9D show, respectively, how signals travel through layers of textiles 5 assembled in the vertical or horizontal directions. Much like in the example single sheet electrodes shown in FIGS. 9A and 9B, in FIGS. 9C and 9D the signal travels between a network of electrically conductive and semi-conductive yarns protected by electrically non-conductive regions and/or sacrificial electrically conductive regions. Such electrically non-conductive and/or sacrificial regions are designed to reduce susceptibility of the signal-transmission layer(s) to parasitic capacitive and/or inductive noise, as well as to provide protection against RF and/or EM interference. In addition, the assembly of electrodes can be designed to have lower sensitivity to mechanical changes (e.g., deformation, such as is caused by bending, stretching, twisting, and/or pressure) through a network of cut, folded, sewn, and/or stacked electrodes.

Electrodes can be made into assemblies of individual electrodes that are cut, folded, sewn, and/or stacked in the horizontal and/or vertical directions. In the first example embodiment shown in FIG. 9C, electrodes are stacked vertically through cutting and/or folding textiles 5 to form the final electrode. The signal S travels through the width of the electrode by length l. These textiles 5 can be stacked at an angle relative to each other, such that each textile 5 has its own interlaced pattern of yarns offset from that of any neighboring (e.g., immediately adjacent) layers. This offset angular stacking form angled paths in the Z-direction with gaps of electrically conductive, semi-conductive, and non-conductive regions that alternate in repeated patterns. Depending on the relative angular offset between adjacent textile 5 layers, loops N can be formed from cross-hatched yarns (e.g., extending predominantly in the X-Y plane) in the Z direction. These looped patterns can advantageously be used to attract parasitic inductance, thereby eliminating any external inductance signal from the main signal path. However, undesired loops can inadvertently introduce an inductance effect into the signal S if the signal were to travel within these looped regions in an unintended manner. There are also capacitive effects between electrode layers, which are determined by the conductive area covered between each electrode face A, as well as the distance between adjacent layers d. The intersecting faces can be controlled by positioning the stacked layers at angles from one another. Such capacitive regions can attract parasitic capacitance.

Electrodes can be assembled in the horizontal direction by weaving, knitting, sewing, and/or binding yarn between the finished edge of each electrode. The signal S then travels through the axial direction of the textile 5 by length l. An edge of an electrode assembly can further be woven, knitted, sewn, and/or bound to another electrode or electrode assembly to continue the chain. The final edge of the assembled electrode can be referred to as a selvedge edge. These intermediate edges can introduce loops N that produce an inductive effect within the textile 5 in the axial direction. These looped patterns can act to attract parasitic inductance, thereby eliminating any external inductance signal from the primary signal path. In addition, there are capacitive effects between the assembled electrodes at spacing d, with the axial area covered by A. Such capacitive regions can attract parasitic capacitance.

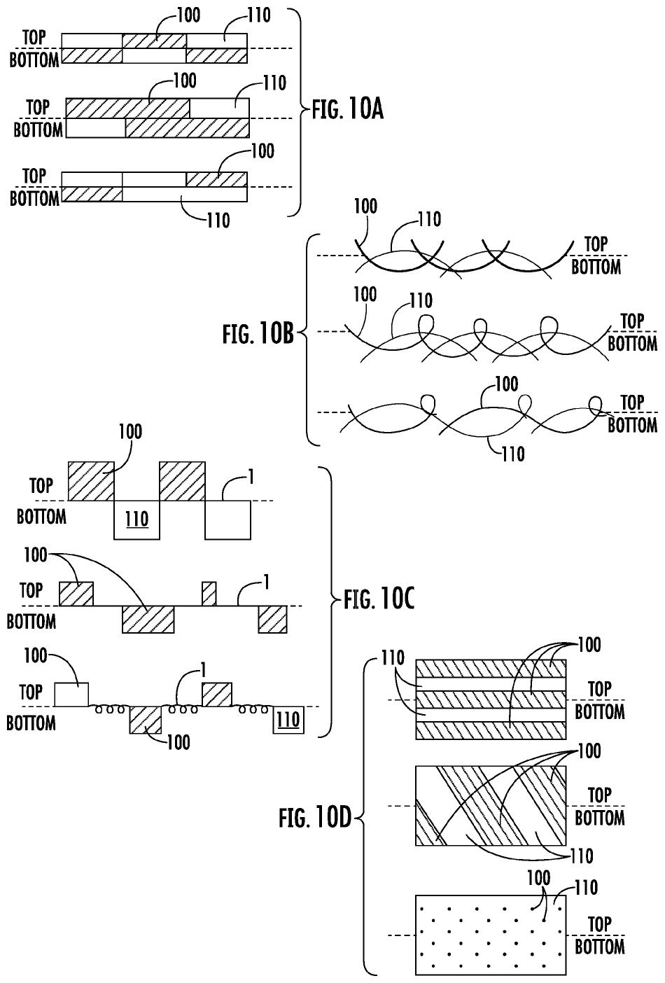

FIGS. 10A-10D show cross-sectional views of textiles made through a woven, knitted, laced, and/or non-woven process. These interlacing methods can advantageously be used to control how a signal flows in the X-, Y-, and Z-directions within an electrode. The signal flow can be transmitted between the top and/or bottom layers, or can be transmitted through an alternating route having top and bottom directions in the z-direction. This control is used to direct signal and to filter out noise. Differential noise is reduced when signals travel in the same direction, while common noise is reduced when signals go through twisted or repeated patterns. Finally, regions of electrically conductive or semi-conductive regions in the interlaced pattern can create islands or reservoirs to absorb or maintain any unwanted signal (e.g., noise) from the signal-transmission yarn(s). The absorption of such noise can be used to reduce externally-generated RF and/or EM interference, as well as to collect and dissipate stray parasitic capacitance and/or inductance from the system and/or from the environment.

FIG. 10A shows cross-sections of example embodiments of a woven pattern made from plain, twill, and satin weaves, respectively, in which the top face and bottom face of the textile expose different regions of electrically conductive material 100 and electrically non-conductive material 110. The top woven pattern is formed using a plain weave, the middle woven pattern is formed using a twill weave, and the bottom woven pattern is formed using a satin weave. FIG. 10B shows a cross-section of example embodiments of a knitted pattern, in which the ribs and courses (i.e., the vertical and horizontal direction of yarns, respectively) alternate between an electrically conductive material 100 and an electrically non-conductive material 110, based on the type of knit. FIG. 10C shows cross-sections of example embodiments of laced textiles, in which segments of dense yarn areas 100, 110 are separated from each other by lengths of a connecting yarn 1. The segments of dense yarn areas 100, 110 can be laced with different combinations and/or densities of electrically conductive or electrically non-conductive yarns 1 to produce a heterogeneous pattern of electrically conductive and/or electrically non-conductive segments, or “islands.” FIG. 10D shows a cross-section of example embodiments of a non-woven textile, in which different non-woven manufacturing processes incorporate electrically conductive material 100 and electrically non-conductive material 110 in different layers, patterns, and/or structures. Examples of such non-woven manufacturing processes include layering alternating patterns of electrically conductive material 100 and electrically non-conductive material 110 in the horizontal and/or vertical directions; interspersing sheets and/or webs of electrically conductive material 100 (e.g., fibers and/or filaments) within a bulk electrically non-conductive material 110; or interspersing sheets and/or webs of electrically non-conductive material (e.g., fibers and/or filaments) within a bulk electrically conductive material.

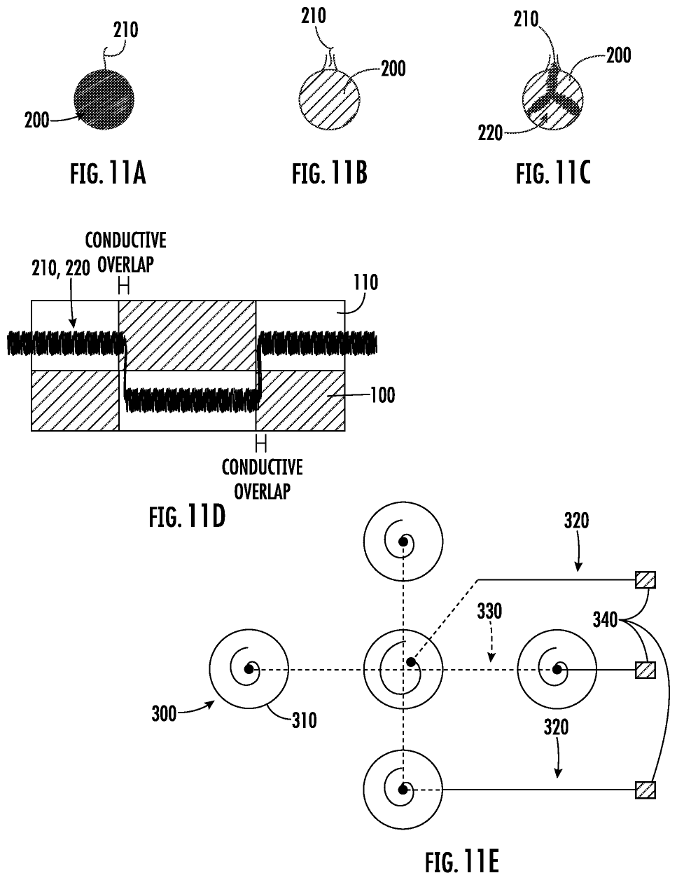

FIGS. 11A-11E show cross-sectional views of example embodiments of textiles that can act as electrodes to receive and transmit signals from the surface of a system. FIGS. 11A-11E show how textiles made from a weaving, knitting, lacing, and/or nonwoven process can be cut, folded, sewn, and/or embroidered to make an electrode. An example embodiment in which embroidering is used to form an electrode is shown in FIG. 11A, in which electrically conductive, semi-conductive, or non-conductive thread 210, yarn, etc. is embroidered onto the surface of an electrically non-conductive or conductive textile 200. Another example embodiment is shown in FIG. 11B, in which an electrically conductive thread 210 is attached by sewing to a terminal end of the textile 200 (e.g., electrode) to interface with a neighboring sewn device. Another example embodiment is shown in FIG. 11C, in which a hybrid of sewing and embroidering is used; thus, a specific conductive, semi-conductive, or non-conductive pattern 220 is embroidered onto an electrically conductive textile using, at least in part, an electrically conductive thread 210 to form a signal path within the textile 200 (e.g., electrode). Embroidery provides additional control as to the types of electrically conductive, semi-conductive, or non-conductive material is/are integrated into an electrode; embroidery can also aid in controlling the directionality of signal flow, which is advantageous for designing a differential and/or common noise rejection mechanism, as well as for absorbing parasitic capacitive, inductive, RF and/or EM noise away from the primary signal path. Using embroidery, it is possible to control tension applied to a top electrically conductive or non-conductive thread and a bottom electrically conductive or non-conductive thread to form a stitch that preferentially routes a signal to the top layer of the electrode, the bottom layer of the electrode, and/or alternating between the top and bottom layers of the electrode. Embroidery can also be used to form patterns to control which regions a signal interfaces within the electrode. These patterns further control how noise is shed from the signal source and absorbed into other regions of the electrode.

FIG. 11D shows a cross-sectional view of a hybrid embroidered-textile electrode containing both electrically conductive material 100 and electrically non-conductive material 110. The textile is made through a woven or knitted pattern 220, which is formed using threads 210, each of which can be electrically conductive, semi-conductive, or non-conductive, but at least one of which is electrically conductive in the example embodiment shown in FIG. 11D. The horizontal and vertical threads 210 in the pattern 220 alternate in electrically conductive and electrically non-conductive materials due to overlays and underlays of yarns. In addition, the tension applied to an electrically conductive thread 210, used as either the top thread or the bottom thread, is controlled by the sewing machine; by controlling the tension, the electrically conductive thread 210 can be moved to the top layer or the bottom layer of the electrode. There may, in some embodiments, be regions of conductive overlap between the embroidered electrically conductive thread 210 and the interlaced yarns. Such conductive overlap regions can be deliberately formed for the signal traveling through the embroidered electrically conductive thread 210 to be transmitted from the electrically conductive embroidered thread 210 to the regions within the textile in which the electrically conductive material 100 is located. Multiple regions of conductive overlap can be used to advantageously enhance distribution of a signal across select regions in the electrode to shed differential and/or common noise. This can also be used for transmitting noise due to parasitic capacitance, inductance, RF, and/or EM interference into regions of the electrode that can absorb and dissipate such noise or otherwise to act as shielding areas away from the primary signal source.

FIG. 11E shows an example embodiment in which a combination of embroidery, conductive textiles, and sewn paths can form a sophisticated architecture of electrodes and connectors that can measure inputs at multiple areas on a two- or three-dimensional (2D/3D) surface. The combination of electrodes, sewn threads, connectors, and an interface to a CPU is referred to herein as a device. The device can be used to measure signals received at the surface of the textile, such signals being useful for such modalities and, for example, EKG, EMG, EOG, EEG, BIA, EDA, and/or SIA. Each circular region is a sensitive pattern 310 within a sensitive area 300, such sensitive areas 300 acting as the electrodes and being made from electrically conductive textiles of interlaced yarns. The sensitive pattern 310 is embroidered within the sensitive area 300 at the electrode to control the area and transmission path of the signal within the electrode. The electrodes are connected, at the end of each of the sensitive pattern 310, via sewn electrically conductive thread(s) 320, 330 that transmit the signal to another region of the surface by transmitting the signal to the top layer or the bottom layer of the device. The sewn thread can go over or under an electrode to avoid cross-talking or shorting between electrically conductive materials used in forming the sensitive patterns 310 and the sewn thread(s) 320, 330. In some embodiments, the sewn thread 320, 330 can be positioned to intersect with an electrically conductive electrode or embroidered region to share and transmit a signal. The signals are consolidated outside of the electrode network in order to bond or attach with connectors 340 that interface with a CPU or another set of electrodes connected elsewhere within the system.

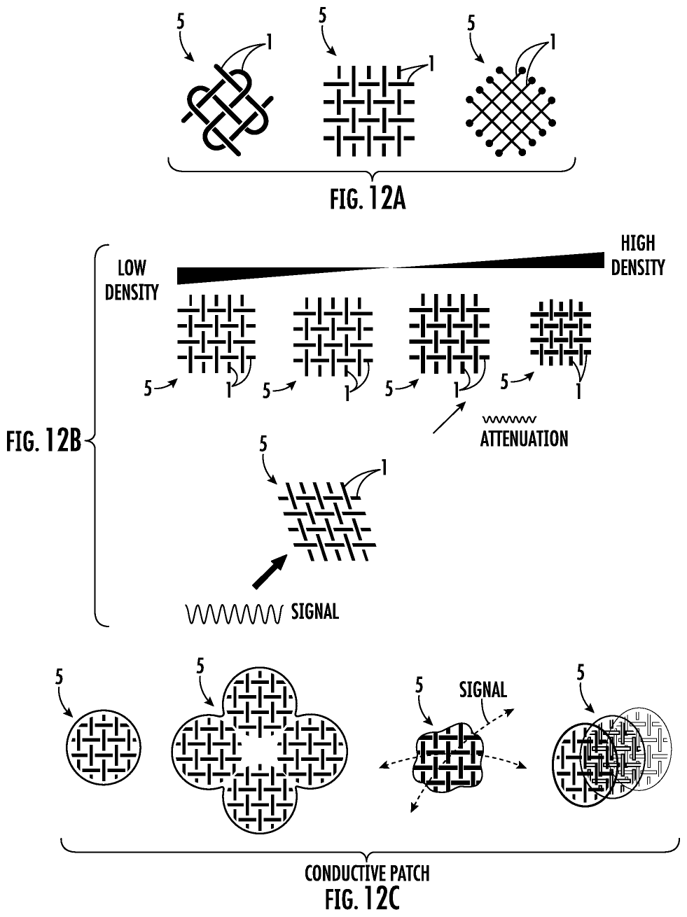

FIGS. 12A-12C show how different yarn and textile types may be utilized in an electrode to optimize for signal-to-noise ratio for signal transmission. The electrodes can also be cut, folded, laced, and/or overall shaped to promote the highest degree of signal transmission while reducing susceptibility to motion-induced noise and motion artifacts. FIG. 12A shows how different woven and knitted textiles can control density of conductive material, and areas of conductive material that can interface with a system.

FIG. 12B shows how the makeup of the yarn 1, such as its thickness, weight, and density of electrically conductive material within or around the yarn, in addition to the weave and knit density, can impact the signal transfer characteristics, also known as the transfer function or impulse response, of an electrode. Features of an electrode in the form of a textile 5 suitable for being optimized by control of aspects of the electrode include, for example, response time, slew rate, bandwidth, dynamic range, settling time, steady-state error, gain, and any desired or undesired low-pass, high-pass, notch, and/or bandpass properties. Signals typically a transmitted through the textile in the transverse direction of the textile, so signals enter from one surface of the yarn and exit from the opposite surface. The signal typically is transmitted through a network of yarns that are organized in such a way that any differential and/or common noise received is rejected through an underlay and/or overlay of electrically conductive yarns. Depending on the pattern, as well as the density of the yarn, signal(s) can be attenuated while traveling through the yarn. Additionally, stacking of textiles can be used to provide further attenuation of the signal due to the increased thickness of the textile assembly. However, the noise is also similarly reduced by being filtered as the signal is transmitted through the network of yarns.

FIG. 12C shows example embodiments of how an electrode can be cut, folded, sewn, embroidered, and/or stacked horizontally or vertically with neighboring (e.g., adjacent, including directly adjacent) electrodes to produce an electrode assembly. As shown, a single electrode can be cut and folded into a shape that allows the surface contact of the electrode with the system to increase regardless of movement or surface distortion of the electrode from the system. The importance of maintaining surface contact of the electrode is to maintain impedance matching, which generates the greatest power transfer between interfacing layers. For surfaces that have curved 3D shapes, planar electrodes can be cut and/or folded in an origami and/or kirigami pattern such that the electrode can bend, stretch, and/or deform along the curved contours and/or lines of such curved 3D shape. Electrodes can also be cut to form so-called cloverleaf or repeated patterns to provide enhanced redundant contact of the electrode on the surface, such that at least one point of contact is maintained during deformation of the surface; such patterns can also contain cuts and folds to allow multiple appendages of the electrode to touch multiple areas of the sensing area of interest to further add redundancy through fractal patterns. The electrode may also be thickened by adding embroidered patterns, sewing cushion or insulating layers, and/or stacking electrodes on top of each other to enhance signal transfer in a prescribed direction. For example, by sewing a cushion or insulating layer on a region of the electrode, the signal must be transmitted around the insulating region in proceeding to the next closest electrically conductive region. Similarly, biased sewing or embroidery, in which a pattern is sewn with a specific direction, can also be used to guide signals from one region of the electrode to another. The ability to control signal orientation and directionality can advantageously be used for filtering out differential and/or common noise from the signal as the signal passes through the electrode, as well as aiding in the absorption or filtering of parasitic capacitive, inductive, RF, and/or EM interference from the signal.

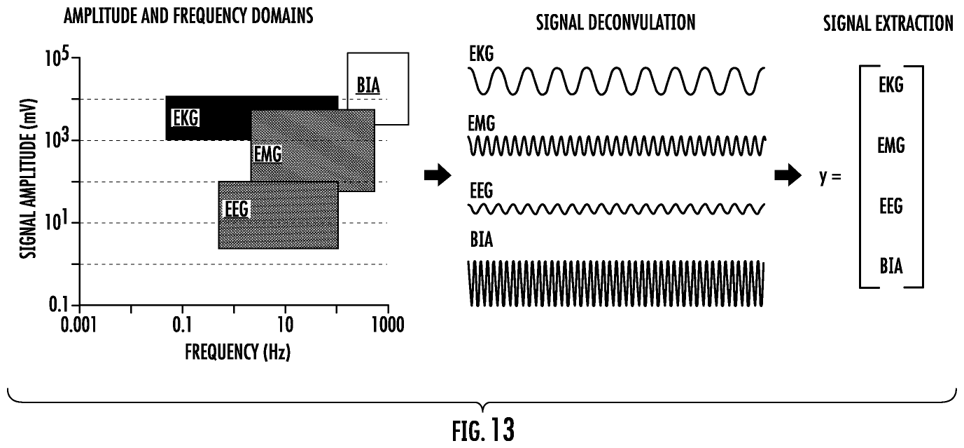

FIG. 13 is a graphical representation of how a single electrode can differentially measure signals received from the body (e.g., a human body). Signals from the body that are suitable, for example, use in EEG, EOG, EKG, EMG, BIA, SIA, EDA, and/or any other suitable modality that can be performed using skin-based measurements can be picked up from a single electrode or sets (e.g., pairs) of electrodes. The ability of the presently disclosed subject matter to simultaneously measure multiple modalities is due to the fact that each diagnostic modality has its own signature amplitude range, signal rms (root mean square), noise profile, and frequency range.

An example embodiment of a method of operation of the system is to have the electrode selectively measure a single modality that is then processed by the CPU. This means the electrode selectively filters for the specified modality, disregarding (e.g., filtering) signals that are not associated with the specified modality. According to this method, the CPU and/or the electrode perform noise cancellation and signal extraction through algorithms, including, for example and without limitation, lock-in detection, wavelet-thresholding, empirical mode decomposition, detrended fluctuation analysis, adaptive filtering, etc. Such algorithms are tuned through hyperparameters unique to the specified modality. For example, EKG requires a specific set of noise canceling and wavelet extraction methods, or combinations of methods, whereas EMG requires a different specific set of noise cancellation and extraction methods. These individual steps can be quickly cycled such that the electrode can measure and/or receive signals associated with all compatible modalities sequentially without a loss of information associated with any of the modalities. The cycling of the electrode, also known as the sampling rate, must be equal or higher than twice the Nyquist frequency of the signal. The Nyquist frequency is defined as the highest frequency to reconstruct a signal without aliasing or other artifacts. Thus, as a merely illustrative example, it will be presumed for the sake of this example that the typical frequency range of a heart rate is 1-100 Hz and, accordingly, the Nyquist frequency should be 100 Hz to fully capture this signal bandwidth. Therefore, the sampling rate of the electrode should be at least twice the Nyquist frequency, or 200 Hz or more.

Another method of operation of the system includes multiplexing signal acquisition for each modality all at once (e.g., simultaneously). Thus, according to this method, the raw signal is received and/or measured, then decomposed for each modality by the CPU, instead of sequentially extracting each modality from the raw signal at the time of measurement and disregarding information from modalities not being measured at the moment the signal is received. This simultaneous measurement method is more direct, but requires greater computational resources. When the entirety raw signal is received and/or measured at the electrode, the frequency and amplitude information is used to deconvolve the signal associated with each modality from the signals associated with the other modalities. Deconvolution algorithms rely on the Fourier Transform, a broader definition of which is the Laplace Transform. In the Fourier Transform, signals are converted into the frequency domain to form a transformed signal. The transformed signal is then sampled, segmented, and filtered to select ranges of frequencies, or frequency segments, that can be used for performance of one of the specified modalities. Each of the specified modalities has its own frequency signature in this domain. The frequency segments are extracted and transformed back into the signal, or spatial, domain where the signal portion associated with each of the specified modalities are now separated from each other. These signal portions can be further filtered to remove any common noise or gain any low-signal measurements. Each signal portion associated with one of the specified modalities is organized into a matrix, where the amplitude and extracted frequencies of such signal portions are linearly decomposed to output a vector containing the desired metrics for each of the specified modalities. Metrics can be, for example, heart rate for EKG, intensity of muscle activity for EMG, water weight for BIA, and the like.

Modalities such as BIA, SIA, and, in some instances, EDA can be separated from the signal processing for EKG, EMG, EEG, EOG and the like. The reason for this is that BIA, SIA and EDA first require an initial stimulus to enter the system (i.e. body) via one or more electrodes before a response measurement can be acquired from another one or more electrodes. For example, BIA requires a voltage and current to be transmitted into the system, which is then measured on an opposing electrode. The signal and frequency differential between the input and output measurement is what determines metrics. such as body fat content, water weight, etc. Therefore, the post-processing multiplex analysis for EKG, EMG, EOG, and EEG can be performed separately from the signal acquired from BIA, SIA, and EDA which makes the computational intensity associated with deconvolving signals less intense for a CPU that can thread multiple operations in parallel.

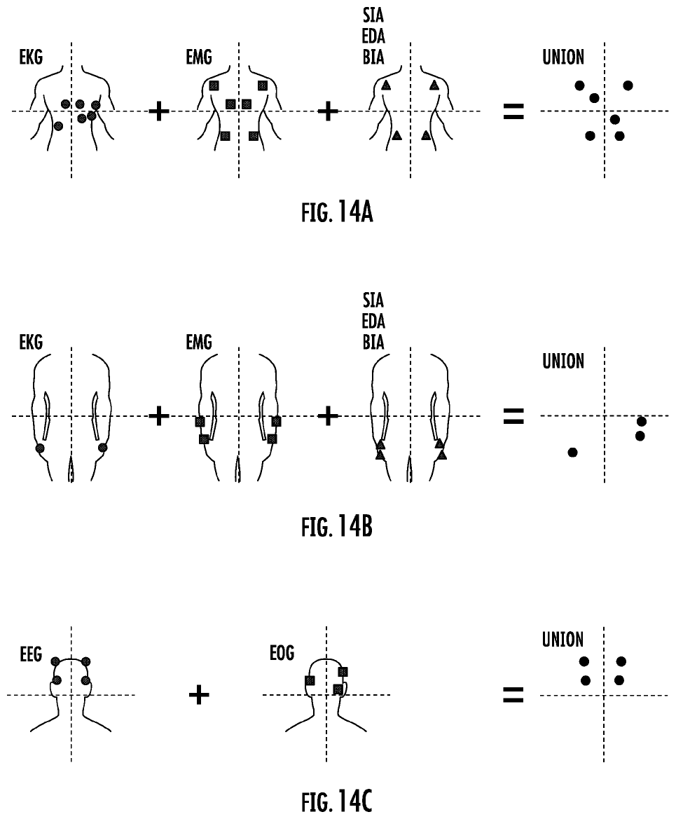

FIGS. 14A-14C show example embodiments for groups of electrodes in a system that may be organized into a set of electrodes to enable multiplex detection of multiple skin-voltage measurements on the human body at the same time (e.g., simultaneously), for example EEG, EOG, EKG, EMG, BIA, SIA and EDA, and other derivative types of skin and surface-based measurements (also referred to herein as a modality). When creating a set of electrodes, the quantity of electrodes placed on the surface are advantageously minimized, as is the total surface area covered (e.g., spanned) by the electrodes, while optimizing placement of such set of electrodes for improved signal detection and specificity. This set of electrodes can be referred to as an optimized electrode set. Optimized electrode sets can be determined empirically. Experimentally, separate electrode sets are determined for each modality. For example, for a requirement to perform modalities of EKG, EMG, and BIA, there would be three separate electrode sets determined, one for each modality. Next, the electrode set with the most stringent requirement is selected. A stringent requirement can be an electrode set with the highest quantity of electrodes required for performing the modality, sizes and shapes required for one or more electrodes of the electrode set, specific placement of each electrode on the body, etc. The electrode set with the most stringent requirement is then used to receive and measure a signal for the other modalities designated to be performed. For example, if the most stringent electrode set is for EKG, the results for EMG and BIA would also be measured using the electrode set for the EKG modality. The signal quality is then assessed for the other specified modalities and an assessment is made as to whether or the electrode set for the EKG modality needs to be modified to provide an optimal position, in total, for ensuring adequate signal quality for all of the specified modalities. Electrodes in the selected electrode set for one modality can be reshaped and/or moved to enhance the signal quality of the other electrodes. After the electrode set has been modified for the other selected modalities, it is necessary for the original modality, in this case EKG, to be performed again to ensure no appreciable loss of signal quality has occurred from the modifications to the selected electrode set. These steps (e.g., of modifying shape and placement of electrodes) are executed iteratively per electrode set until there is no further improvement to be gained for any modality and/or until the signal loss for an electrode set is below the requirements for the original selected modality associated with the electrode set.

A further evaluation that may be performed for an electrode set is evaluation of eliminating one or more electrodes in an electrode set. The minimum requirement is two electrodes; however, the use of a higher quantity of electrodes would result in improved signal quality and specificity. For example, if the requirement for a specified modality is to measure a specific signal direction (e.g., muscle movement in the sagittal, frontal, transverse, or angled combinations of these planes) then at least two electrodes are needed at the start and end of the vectored direction to match the directional measurement line of interest. When multiple modalities are measured, the line of best fit between each modality's direction can be calculated to define the vector of the start and end electrode, producing two electrodes to measure multiple modalities. In instances where a regression analysis of the measured vector direction for each modality does not exist, or produces a measurement below the quality required, a third electrode can be introduced to supplement data capture and/or decrease noise. This method can be repeated until the appropriate minimum number of electrodes are determined to measure all directions needed for each modality specified to be measured.

A further method comprises using algorithmic models to identify optimal electrode sets for a chosen set of modalities. Algorithms such as supervised, unsupervised, and/or reinforced machine models, can be used to determine an optimized electrode set. A first of the method is to supply these models with statistical parameters of an electrode, such as its X, Y, and Z placement coordinates, signal intensity, noise level, and any relevant electrical signal parameters. These parameters, also known as features, are extracted for each electrode and for each modality. These features can be further assembled into engineered features, in which linear or non-linear combinations of these features are condensed into an aggregated set through dimensional reduction. Examples of such feature reduction methods are principal component analysis (PCA), lasso, factor analysis, linear discriminant analysis, singular value decomposition, kernel PCA, stochastic neighbor embedding, multidimensional scaling, isometric mapping, etc. Features per electrode per modality are then assembled into a matrix and input into a machine model where the objective function is to reduce the number of electrodes, optimize for X, Y, and Z placement coordinates, and optimize signal per electrode for each modality. Examples of unsupervised models include hierarchical clustering, k-means clustering, nearest neighbor (NN), and k nearest neighbors. Examples of supervised models include linear regression, logistic regression, support vector machines, decision trees, neural nets, etc. These algorithms can be modified to become semi-supervised or reinforced models. Once these models are trained on the statistical parameters for each electrode and for each modality, these trained models can be modified to become generative or discriminative models to algorithmically produce an optimized electrode set. The output of such models is a table of electrodes, with their features listed as columns, for example, X, Y, Z placements, size, and shape. The quantity of electrodes listed in the table of electrodes is the optimal quantity of electrodes for measuring all of the selected modalities.

FIG. 14A shows how empirical testing or execution of an algorithm can be used to determine the optimal electrode set for EKG, EMG, BIA, EDA, and SIA on the torso of a body. An example use case is providing a compression shirt, vest, bra, or strap with electrodes embedded therein that can be worn over the body of the user for performance of these modalities. The optimal electrode set is shown on the right side of FIG. 14A. FIG. 14B shows how empirical testing or execution of an algorithm can be used to determine the optimal electrode set for EKG, EMG, BIA, EDA, and SIA on the arms and wrists of a body. An example use case is providing a long sleeve shirt, gloves, or wrist straps with electrodes embedded therein. The optimal electrode set is shown on the right side of FIG. 14B. FIG. 14C shows how empirical testing or execution of an algorithm can be used to determine the optimal electrode set for EEG and EOG, particularly targeted around the eyes, skull, and cranium. The optimal electrode set is shown on the right side of FIG. 14C.

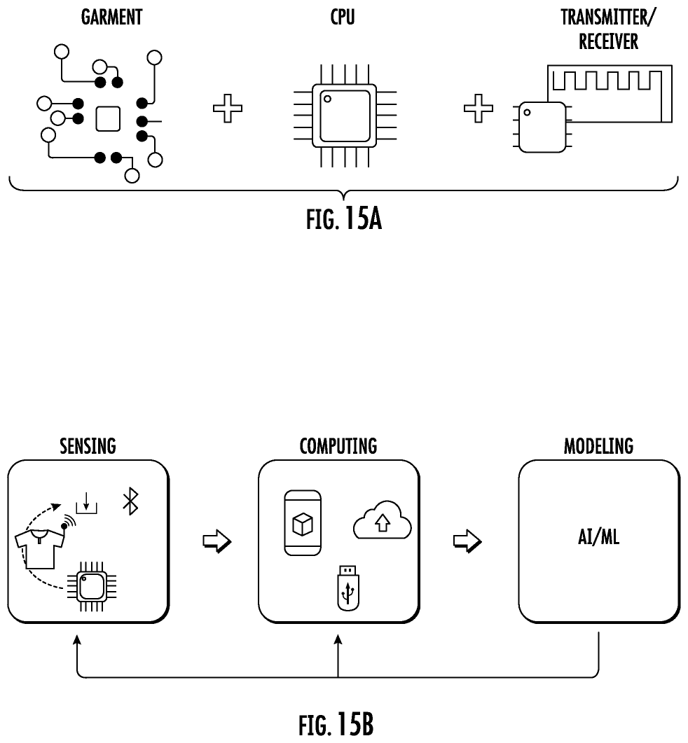

FIGS. 15A and 15B show example embodiments for the components of a measurement system, as well as the pipeline in acquiring, filtering, storing, and processing received data. FIG. 15A shows the system comprising a garment, which is used as the sensor, a CPU that processes the data, and a transmitter and receiver (e.g., a transceiver) that receives the data. In this example embodiment, the garment contains fibers, filaments, yarns, textiles, threads, and/or connectors that interface with the CPU and transceiver. The body on which the garment is worn generates surface electromechanical signals such as current, voltage, resistance, capacitance, and/or inductance, which values are transmitted through the electrically conductive and/or semi-conductive threads and/or connecting wires of the garment to connectors that interface with the CPU, which also in some embodiments contains peripheral components such as a battery, voltage regulators, and components necessary to control power usage and communication protocols for the CPU. The CPU then interfaces with a transceiver (e.g., in a wired manner, such as via a USB or coaxial cable, or in a wireless manner, such as via Bluetooth® and/or WiFi®). Processed data from the CPU is then converted into a signal stream that can be emitted through radio frequencies from the transceiver to a receiving device.

FIG. 15B shows the overall pipeline in which a garment is used to acquire signals from a body for machine learning applications. In a first step, the garment senses a signal from the body using the sensors embedded in the garment. In a next step, the signal is transferred to a CPU to analyze and process the signal into data. In a next step, the data is optionally stored directly in the garment or in the CPU itself, such as in flash, eeprom, ram, rom, or masked memory. In a next step, the data is transmitted via wired or wireless communication protocol to a receiving device, which can be in some embodiments a separate storage device, such as a flash drive, dedicated hard drive, computer, and/or a personal device such as a phone, tablet, watch, and/or laptop. In a next step, the data can be further processed, plotted, and presented to a user via an app or other graphical user interface (GUI). In a next step, the data is stored locally in the receiving device. In a next step, the data can be transmitted to a cloud infrastructure in a dedicated server or server hub, in which the servers consolidate multiple CPUs and GPUs to perform dedicated data operations which can then be transmitted to the personal devices and/or local computers through data transfer protocols, such as HTTP, TCP, FTP, UDP, etc. Once the data is stored, the storage system can then implement machine learning and artificial intelligence algorithms and analytics on the collected dataset. Metrics, insights, and generative reports can be presented to such devices as numbers, reports, warnings, screens, dashboards, etc. that are presented to the user and/or for display by the garment itself (e.g., using audio, LEDs, haptic, visual cues, etc.).

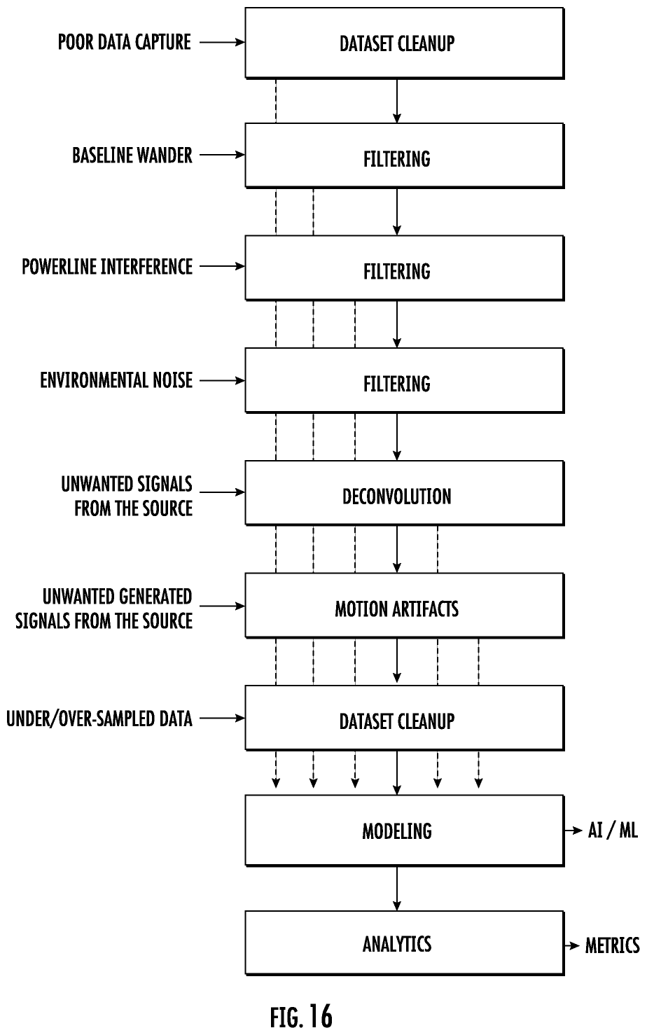

FIG. 16 shows an example embodiment of a flowchart for the data transformation and processing steps that allow an electrode to extract and distinguish (e.g., simultaneously) specific signals from multiple modalities, such as EKG, EMG, EOG, EEG, BIA, EDA, and/or SIA. These steps can be performed in real-time as signals are acquired by the electrodes or post-processed once data collection for the modalities is complete. Text on the left of FIG. 16 is used to describe the type of data and/or analytical problem that is experienced at each stage and the stage itself describes the operation and/or algorithm used to process the data and/or remedy the analytical problem. Arrows indicate the next stage that can be performed after a successful operation in the current stage. Dashed arrows indicate how output from one stage might skip subsequent stages because the intermediate data and/or analytical problems may not exist in each scenario.

In one example, a single electrode is used for measuring signals associated with performance of an EKG. In a first step of the method, the dataset is preprocessed, or “cleaned up,” meaning that any outlier values are removed, missing values due to CPU or clock issues are interpolated, and/or timestamps, headers, and/or units are reformatted so that a “clean” dataset is available for use in the next step. The next step of the method comprises correcting any baseline wander and/or fluctuations in the data, which refers to stochastic and/or intermittent deviation(s) of the baseline from the signal's expected average. An example of a filtering technique suitable for eliminating baseline wander is to place a high-pass filter at a low cut-off frequency, typically in the range of about 0.1 Hz to about 10 Hz. This low frequency cut-off is used to remove any system or body motion artifacts such as movement, twisting, turning, and/or translation of the system in space. The next step of the method comprises filtering powerline interference, since most power sources operate by transforming AC power into DC power, either through outlets, plugs, jacks, and/or AC/DC converters. These AC signals can interact with the power sources of the measuring device and CPU, which can introduce a distinct noise in the range of about 50 Hz to about 60 Hz. In some embodiments, a notch filter in this frequency range may be used to provide such filtration of powerline interference.

The next step of the method comprises filtering environmental noise, which can come from RF interference, EM interference, and/or neighboring capacitive or inductive parasitics from nearby devices. Such environmental noise can be filtered through a combination of notch and bandpass filters that selectively eliminate the noise within a specified frequency range (e.g., the frequency range in which a majority of such noise occurs). However, it is vital to ensure that notching and/or blocking the passage of frequencies of the measured signal is avoided.

The next step of the method comprises filtering, eliminating, and/or deconvolving undesired signals (e.g., differential noise) that are detected by the electrode. In the example in which the selected modality is an EKG, it is possible that EMG signals may be detected by the electrode as high frequency signals. These high frequency signals can be selectively eliminated by performing a Fourier Transform and removing any EMG signals and harmonics. Other techniques, such as an N-point moving average filter, time varying low-pass filter, or gaussian impulse response filter, can also be used to remove EMG signals. The filtering steps described as being performed herein depend on the modality being performed and, specifically, the characteristics of the signal detected while performing the selected modality. Thus, while the aforementioned techniques have been described in relation to the performance of an EKG, filtering of a signal associated with performance of an EEG, EOG, BIA, etc. may require different and/or additional filtering techniques to be performed on the signal and/or may use the same filtering techniques but with parameters optimized for the anticipated characteristics of the signal for performance of such selected modality.

The next step of the method comprises eliminating any specific electrode motion artifacts, which are caused when, for example, the electrode bends and/or distorts and/or the yarns within the electrode are disturbed, which can introduce electrode-induced noise as signal. In this step, such electrode-induced noise is disrupted as it is transmitted through the electrode towards the CPU. These motion artifacts and their transfer functions can be determined prior to measurement with sufficient empirical characterization of the system. Electrode signal acquisition behavior can be well-characterized before performance of the modality through empirical and simulation testing of 2D and/or 3D distortion of the electrode. With sufficient data as to the characterization of the electrode, such electrode-specific motion artifacts and their transfer functions can be machine learned and filtered out through, for example, deconvolution, least mean square, recursive mean square, and/or deep learning algorithms, such as neural nets, random forest, and/or decision trees.

While the steps of the method discussed heretofore are used to eliminate undesirable data from the dataset, the next step of the method comprises interpolating or reconstructing any lost signal-specific data that pertains to the performance of the selected modality. Techniques such as interpolation, smoothing, regression, under and/or over-sampling, peak extraction, nearest neighbor searches, and combinations thereof can be used in this step. After reconstruction of the signal associated with the performance of the selected modality, the method comprises a modeling step, in which the signal enters a machine model, which learns which parameters of the signal are of interest and/or extracts the desired signal from the remaining signal provided by the previous stage. These models can be handled through unsupervised, supervised, semi-supervised, and/or reinforced learning models. In the next step of the method, an analytics step, the extracted signals, or parameters of the signals, such as its peak height, average, distance, etc. are provided as an input to a custom algorithm or another machine model to generate metrics that are more readily understood by humans. In this analytics step, human readable plots, reports, and/or actionable feedback is generated that can be used by the user to change the configuration, habits, or ways such person interacts with the device and/or environment.

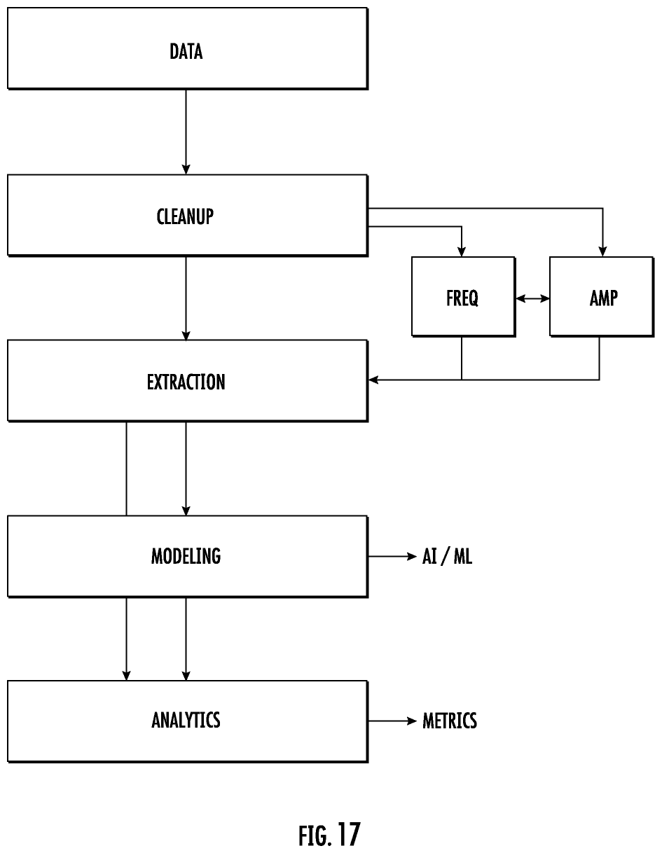

FIG. 17 shows an example embodiment of a flowchart for regarding how a single electrode can be used to simultaneously extract a signal containing information pertinent with the performance of multiple modalities (e.g., simultaneously) from a raw data source. The first step comprises collecting a raw signal from the source. A signal can be a combination of EKG, EMG, EOG, EEG, BIA, EDA, and SIA information, for example. Noise can also be physically eliminated through the electrode itself by over and underlaying yarns, or embroidering paths of electrically conductive and semi-conductive material (e.g., threads) in patterns in the electrode to preferentially occlude noise and transmit the desired signal to the connections and/or the CPU. Additionally, these patterns can be used to differentiate signals from the collective signal by allowing different frequency signals to be transmitted along different paths within the electrode or embroidered areas. Thus, signals are received at the connections and/or to the CPU at different times, frequencies, and/or amplitudes.

When data is received by the connector(s) of the device, data can be “cleaned” using filtering techniques or noise elimination techniques as described with respect to FIG. 16. After the signal has been post-processed to remove a sufficient amount of artifacts, the CPU can extract relevant information from the data directly and/or the data can further be partitioned in the time and/or frequency domains through transformation or decomposition algorithms. Transformation and decomposition algorithms include, for example, the Fourier transform, Laplace transform, discrete wavelet transform, deconvolutions, and other amplitude and/or frequency modulation and demodulation techniques. Since the signals from each of the modalities are segregated from each other, these signals are then transformed into their original signal but are now partitioned from the input signal source with respect to the frequency and amplitude information. In another step of the method, the extracted signals are transmitted as an input to a machine model for training and/or analytics for generating metrics and insights.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate for the disclosed devices, compositions, systems and/or methods.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

It will be understood that various details of the presently disclosed subject matter may be changed without departing from the scope of the presently disclosed subject matter. Furthermore, the foregoing description is for the purpose of illustration only, and not for the purpose of limitation.

1-28. (canceled)

29. An electrically conductive textile-based electrode comprising:
a textile comprising a plurality of yarns interlaced in horizontal, vertical, and/or angled directions;
wherein the plurality of yarns comprises yarns that are electrically conductive, electrically semi-conductive, and/or electrically non-conductive; and
wherein the electrode is configured to form and/or control a primary signal path for transmission of signals in an axial direction and/or in a transverse direction.

30. The electrode of claim 29, wherein the plurality of yarns are formed in repeated or irregular patterns of underlays and overlays that are configured to transmit the signals in a direction of extension of the electrode, as well as on a top surface, internal to, and/or on a bottom surface of the electrode.

31. The electrode of claim 29, wherein the plurality of yarns are assembled together using a weaving technique, a knitting technique, a lacing technique, and/or a non-woven technique to form the electrode.

32. The electrode of claim 29, wherein a shape, size, thickness, and/or material type of the electrode can be selected to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

33. The electrode of claim 29, wherein, in forming the electrode, the textile is cut, folded, sewn, embroidered, and/or stacked horizontally and/or vertically to have a series of textile layers that can each be electrically conductive, electrically semi-conductive, and/or electrically non-conductive.

34. The electrode of claim 33, wherein cutting and/or folding of the textile and/or stacking a series of textile layers horizontally and/or vertically is used to control a primary transmission path for the signals in a direction of extension of the textile and/or in a direction perpendicular to the direction of extension.

35. The electrode of claim 29, wherein, for the textile, ends per inch, picks per inch, stitches per inch, knits per inch, and/or weaves per inch can be selected to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

36. The electrode of claim 29, wherein, for the textile, a weight, a density, a stitch pattern, a ratio of underlay and overlay yarns of the textile and a direction of the signals within the electrode are selected to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

37. The electrode of claim 29, wherein a stitch pattern of the textile from which the electrode is formed can be selected to control a signal transmission path in which the signals can gain or attenuate measurements comprising voltage, current, resistance, capacitance, and/or inductance.

38. The electrode of claim 29, wherein a stitch pattern of the textile from which the electrode is formed can be selected to control a signal transmission path in which the signals can disrupt, shield, and/or absorb external noise from radio frequencies, electromagnetic radiation, and/or voltage, current, resistive, capacitive, and/or inductive signals from an adjacent noise source.

39. The electrode of claim 29, comprising electrically conductive and/or electrically semi-conductive yarns that are embroidered in the textile to control a direction of transmission of the signals within the electrode to aggregate, absorb, or differentially transmit signal and noise sources.

40. The electrode of claim 29, comprising an electrically conductive and/or electrically semi-conductive yarn that is, by varying a tension applied thereto when being sewn into the textile, at the top surface and/or the bottom surface of the textile to control a direction of transmission of the signals within the electrode and/or an interface with electrically conductive, electrically semi-conductive, and electrically non-conductive regions formed in the textile.

41. The electrode of claim 29, wherein, by cutting or folding the textile and/or by stacking a series of textile layers horizontally and/or vertically, the electrode is configured to maintain at least one area of contact, and/or with a fractal pattern, with a measurement location to ensure sufficient impedance matching for signal transmission.

42. The electrode of claim 29, wherein the electrode is configured such that the signals can enter or exit the electrode through a textile patch, which is sewn, embroidered, hemmed, crimped, soldered, magnetic, chemical bond, or combinations thereof to the electrode, to connect the electrode with further devices.

43. The electrode of claim 29, comprising a plurality of horizontally or vertically stacked textile layers formed from the textile, wherein the textile layers are angled such that the horizontal and vertical yarns create looped patterns or pores between the textile layers to form the electrode.

44. The electrode of claim 29, comprising a plurality of horizontally or vertically stacked textile layers formed from the textile, wherein the textile layers are configured to control a resistive signal, a capacitive signal, and/or an inductive signal through a transverse direction of the electrode.

45. The electrode of claim 29, comprising a plurality of horizontally or vertically stacked textile layers formed from the textile, wherein the textile layers are knitted, woven, sewn, and/or electromechanically and/or chemically attached to secure edges of the electrode in repeating patterns, thereby controlling signal transmission within the electrode.

46. The electrode of claim 29, wherein the textile is embroidered, folded, cut, and/or stacked with an additional textile layer configured as a signal reservoir and/or a sacrificial textile layer for absorbing noise.

47. The electrode of claim 29, wherein the textile is embroidered, folded, cut, and/or stacked with an additional textile layer for impedance matching with a measurement location to optimize power transmission into and/or out of the electrode.

48. A method of forming an electrically conductive textile-based electrode, the method comprising:
interlacing a plurality of yarns in horizontal, vertical, and/or angled directions to form a textile, wherein the plurality of yarns comprises yarns that are electrically conductive, electrically semi-conductive, and/or electrically non-conductive;
forming and/or controlling a primary signal path within the electrode; and
transmitting signals along the primary signal path in an axial direction and/or in a transverse direction;
wherein the primary signal path is controlled such that the primary signal path passes through or adjacent to electrically non-conductive regions of the textile and electrically conductive regions of the textile to reduce noise and/or reject transmission of differential and/or common noise; and
optionally, wherein the electrically non-conductive regions and the electrically conductive regions of the textile absorb, in the manner of a reservoir, noise introduced along the primary signal path.

49. The method of claim 48, comprising:
forming the plurality of yarns in repeated or irregular patterns of underlays and overlays; and
transmitting, via the underlays and overlays, the signals in a direction of extension of the electrode, as well as on a top surface and/or on a bottom surface of the electrode.

50. The method of claim 48, comprising assembling the plurality of yarns together using a weaving technique, a knitting technique, a lacing technique, and/or a non-wove technique to form the electrode.

51. The method of claim 48, comprising selecting a shape, size, thickness, and/or material type of the electrode to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

52. The method of claim 48, comprising, while forming the electrode, cutting, folding, sewing, embroidering, and/or stacking the textile horizontally and/or vertically to have a series of textile layers that can each be electrically conductive, electrically semi-conductive, and/or electrically non-conductive.

53. The method of claim 52, wherein cutting and/or folding of the textile and/or stacking a series of textile layers horizontally and/or vertically is used to control a primary transmission path for the signals in a direction of extension of the textile and/or in a direction perpendicular to the direction of extension.

54. The method of claim 48, comprising selecting, for the textile, ends per inch, picks per inch, stitches per inch, knits per inch, and/or weaves per inch to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

55. The method of claim 48, comprising selecting, for the textile, a weight, a density, a stitch pattern, a ratio of underlay and overlay yarns of the textile and a direction of the signals within the electrode to control a response time, an input dynamic range, an output dynamic range, a bandwidth, a signal-to-noise ratio, a common-noise rejection ratio, differential-noise rejection, a signal gain, a sensitivity, and/or an insensitivity of the electrode.

56. The method of claim 48, comprising selecting a stitch pattern of the textile from which the electrode is formed to control a signal transmission path in which the signals can gain or attenuate measurements comprising voltage, current, resistance, capacitance, and/or inductance.

57. The method of claim 48, comprising selecting a stitch pattern of the textile from which the electrode is formed to control a signal transmission path in which the signals can disrupt, shield, and/or absorb external noise from radio frequencies, electromagnetic radiation, and/or voltage, current, resistive, capacitive, and/or inductive signals from an adjacent noise source.

58. The method of claim 48, comprising embroidering electrically conductive and/or electrically semi-conductive yarns in the textile to control a direction of transmission of the signals within the electrode to aggregate or differentially transmit signal and noise sources.

59. The method of claim 48, comprising varying a tension applied to an electrically conductive and/or electrically semi-conductive yarn that is sewn into the textile, at the top surface and/or the bottom surface of the textile to control a direction of transmission of the signals within the electrode and/or an interface with electrically conductive, electrically semi-conductive, and electrically non-conductive regions formed in the textile.

60. The method of claim 48, comprising maintaining, by cutting or folding the textile and/or by stacking a series of textile layers horizontally and/or vertically, at least one area of contact, optionally, with a fractal pattern, with a measurement location to ensure sufficient impedance matching for signal transmission.

61. The method of claim 48, comprising transmitting the signals into or out of the electrode through a textile patch, which is sewn, embroidered, hemmed, crimped, soldered, magnetic, chemical bond, or combinations thereof to the electrode, to connect the electrode with further devices.

62. The method of claim 48, comprising:
forming textile layers from the textile;
stacking, horizontally or vertically, the textile layers; and
angling adjacent textile layers relative to each other such that the horizontal and vertical yarns create looped patterns or pores between the textile layers to form the electrode.

63. The method of claim 48, comprising:
forming textile layers from the textile;
stacking, horizontally or vertically, the textile layers; and
using the textile layers to control a resistive signal, a capacitive signal, and/or an inductive signal through a transverse direction of the electrode.

64. The method of claim 48, comprising:
forming textile layers from the textile;
stacking, horizontally or vertically, the textile layers; and
knitting, weaving, sewing, and/or electromechanically and/or chemically attaching the textile layers to secure edges of the electrode in repeating patterns, thereby controlling signal transmission within the electrode.

65. The method of claim 48, comprising embroidering, folding, and/or stacking the textile with an additional textile layer, which is operable as a signal reservoir, and/or a sacrificial textile layer, which is operable for absorbing noise.

66. The method of claim 48, comprising embroidering, folding, and/or stacking the textile with an additional textile layer for impedance matching with a measurement location to optimize power transmission into and/or out of the electrode.

67-96. (canceled)
